SEQUENCE LISTING (22 sequences)

SEQ ID NO: 1 - protein, 9 aa, synthetic construct
ASNENMETM

SEQ ID NO: 2 - protein, 15 aa, synthetic construct
QVYSLIRPNENPAHK

SEQ ID NO: 3 - protein, 551 aa, synthetic construct
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLSQSYINDKGKEVLVLWGIHHPSTTADQQSLYQNADAYVFVGTSRYSKKFKPEIAIRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVGTGYIPEAPRDGQAYVRKDGEWVLLSTFLGS

SEQ ID NO: 4 - protein, 551 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGS

SEQ ID NO: 5 - protein, 573 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNSSFFSRLNWLTHLNYKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGSGLNDIFEAQKIEWHEGHHHHHH

SEQ ID NO: 6 - protein, 573 aa, synthetic construct
MKTIIALSYILCLVFAQKLPGNDNSTATLCLGHHAVPNGTIVKTITNDQIEVTNATELVQSSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLNFKYPALNVTMPNNEQFDKLYIWGVHHPGTDKDQIFLYAQASGRITVSTKRSQQAVIPNIGSRPRVRNIPSRISIYWTIVKPGDILLINSTGNLIAPRGYFKIRSGKSSIMRSDAPIGKCNSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVEGYIPEAPRDGQAYVRKDGEWVLLSTFLGSGLNDIFEAQKIEWHEGHHHHHH

SEQ ID NO: 7 - protein, 495 aa, synthetic construct
MPMGSLQPLATLYLLGMLVASVLSAHHHHHHGSGSLVPRGSPSRSIINETADDIVYRLTVIIDDRYESLKNLITLRADRLEMIINDNVSTILASIGSGTGVAGQDVTSVKLAGNSSLCPISGWAIYSKDNSIRIGSKGDVFVIREPFISCSHLECRTFFLTQGALLNDKHSNGTVKDRSPYRTLMSCPIGEAPSPYNSRFESVAWSASACHDGMGWLTIGISGPDNGAVAVLKYNGIITDTIKSWRNNILRTQESECVCVNGSCFTIMTDGPSNGQASYKIFKIEKGKIVKSVELNAPNYHYEECSCYPDTGIVMCVCRDNWHGSNRPWVSFNQNLDYQIGYICSGVFGDNPRPNDGEGSCGPVTVDGANGVKGFSFKYGNGVWIGRTKSNSSRSGFEMIWDPNGWTGTDSDFSVKQDIIAITDWSGYSGSFVQHPELTGLDCIRPCFWVELIRGLPKENTIWTSGSSISFCGVNSDTANWSWPDGAELPFTIDK

SEQ ID NO: 8 - protein, 493 aa, synthetic construct
MPMGSLQPLATLYLLGMLVASVLSAHHHHHHGSGSLVPRGSPSRSIINETADDIVYRLTVIIDDRYESLKNLITLRADRLEMIINDNVSTILASIGTGKEICPKLAEYRNWSKPQCKITGFAPFSKDNSIRLSAGGDIWVTREPYVSCDPDKCYQFALGQGTTLNNRHSNDTVHDRTPYRTLLMNELGVPFHLGTKQVCIAWSSSSCHDGKAWLHVCITGDDENATASFIYNGRLVDSIGSWSKKILRTQESECVCINGTCTVVMTDGSASGRADTKILFIEEGKIVHISPLSGSAQHVEECSCYPRYPGVRCVCRDNWKGSNRPIVDINVKDYSIVSSYVCSGLVGDTPRKNDSSSSSHCLNPNNEEGGHGVKGWAFDDGNDVWMGRTISEKLRLGYETFKVIEGWSKPNSKLQINRQVIVERGNRSGYSGIFSVEGKSCINRCFYVELIRGRKQETEVWWTSNSIVVFCGTSGTYGTGSWPDGADINLMPI

SEQ ID NO: 9 - protein, 566 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNSSFFSRLNWLTHLNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 10 - protein, 566 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 11 - protein, 566 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 12 - protein, 566 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNSSFFSRLNWLTHLNYKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVELKSGYKDWILWISFAISCFLLCVALLGFIMWACQKGNIRCNICI

SEQ ID NO: 13 - protein, 566 aa, synthetic construct; identical to SEQ ID NO: 9

SEQ ID NO: 14 - protein, 566 aa, synthetic construct; identical to SEQ ID NO: 12

SEQ ID NO: 15 - protein, 573 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNSSFFSRLNWLTHLNFKYPALNVTMPNNEQFDKLYIWGVHHPGTDKDQIFLYAQSSGRITVSTKRSQQAVIPNIGSRPRIRNIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGSGLNDIFEAQKIEWHEGHHHHHH

SEQ ID NO: 16 - protein, 573 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRGSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGSGLNDIFEAQKIEWHEGHHHHHH

SEQ ID NO: 17 - protein, 573 aa, synthetic construct
MKTIIALSYILCLVFAQKIPGNDNSTATLCLGHHAVPNGTIVKTITNDRIEVTNATELVQNSSIGEICDSPHQILDGENCTLIDALLGDPQCDGFQNKKWDLFVERSKAYSNCYPYDVPDYASLRSLVASSGTLEFKNESFNWTGVTQNGTSSACIRGSSSSFFSRLNWLTHLNYTYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGSGLNDIFEAQKIEWHEGHHHHHH

SEQ ID NO: 18 - protein, 573 aa, synthetic construct
MKTIIALSYILCLVFAQKIPGNDNSTATLCLGHHAVPNGTIVKTITNDRIEVTNATELVQNSSIGEICDSPHQILDGGNCTLIDALLGDPQCDGFQNKKWDLFVERSRAYSNCYPYDVPDYASLRSLVASSGTLEFKNESFNWTGVTQNGTSSACIRGSSSSFFSRLNWLTHLNYTYPALNVTMPNKEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHNVYRDEALNNRFQIKGVEGYIPEAPRDGQAYVRKDGEWVLLSTFLGSGLNDIFEAQKIEWHEGHHHHHH

SEQ ID NO: 19 - protein, 579 aa, synthetic construct
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFINYEELREQLSSVSSFERFEIFPKTSSWPNHDSNKGVTAACPHAGAKSFYKNLIWLVKKGNSYPKLSQSYINDKGKEVLVLWGIHHPSTTADQQSLYQNADAYVFVGTSRYSKKFKPEIAIRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYGSGYIPEAPRDGQAYVRKDGEWVLLSTFLGLNDIFEAQKIEWHEGHHHHHH

SEQ ID NO: 20 - protein, 579 aa, synthetic construct
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLSQSYINDKGKEVLVLWGIHHPSTTADQQSLYQNADAYVFVGTSRYSKKFKPEIAIRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYGSGYIPEAPRDGQAYVRKDGEWVLLSTFLGLNDIFEAQKIEWHEGHHHHHH

SEQ ID NO: 21 - protein, 579 aa, synthetic construct
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYGSGYIPEAPRDGQAYVRKDGEWVLLSTFLGLNDIFEAQKIEWHEGHHHHHH

SEQ ID NO: 22 - protein, 579 aa, synthetic construct
MKAILVVLLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSNKGVTAACPHAGAKSFYKNLIWLVKKGNSYPKLNQTYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYGSGYIPEAPRDGQAYVRKDGEWVLLSTFLGLNDIFEAQKIEWHEGHHHHHH

(19) United States
(12) Patent Application Publication
(10) Pub. No.: US 2024/0316178 A1
(43) Pub. Date: Sep. 26, 2024

(54) RECOMBINANT PROTEIN VACCINES FORMULATED WITH ENANTIO-SPECIFIC CATIONIC LIPID R-DOTAP AND METHODS OF USE THEREOF

(71) Applicant: PDS BIOTECHNOLOGY CORPORATION, Princeton, NJ (US)

(72) Inventors: Frank Bedu-Addo, Stamford, CT (US); Joseph Dervan, Doylestown, PA (US); Gregory Conn, Madrid (ES)

(21) Appl. No.: 18/382,979

(22) Filed: Oct. 23, 2023

Related U.S. Application Data
(60) Provisional application No. 63/418,381, filed on Oct. 21, 2022.
(60) Provisional application No. 63/539,066, filed on Sep. 18, 2023.

(51) Int. Cl. A61K 39/145; A61K 39/00

(52) U.S. Cl. CPC A61K 39/145; A61K 2039/55555; A61K 2039/572; A61K 2039/575

(57) ABSTRACT

Provided herein are vaccine compositions including recombinant protein antigens derived from computationally optimized broadly reactive influenza antigen (COBRA) proteins and an immunomodulator, and methods of use thereof. The vaccine compositions include one or more COBRA proteins, and the immunomodulator is a cationic lipid. The cationic lipid includes R-DOTAP. The methods of use of the vaccine compositions includes methods of inducing a humoral immune response against influenza viruses, methods of inducing polyfunctional CD8+ and CD4+ effector T cells against influenza viruses, methods of inducing memory T cells against influenza viruses, methods of enhancing immunity against influenza viruses, and methods of inducing balanced Th1/Th2 immune response against influenza viruses in a subject.

### CROSS-REFERENCE TO RELATED APPLICATIONS

This application claims benefit of priority under 35 U.S.C. § 119(e) of U.S. Provisional Applications No. 63/418,381, filed Oct. 21, 2022, and No. 63/539,066, filed Sep. 18, 2023. The disclosure of the prior applications is considered part of and is herein incorporated by reference in the disclosure of this application in its entirety.

### INCORPORATION OF SEQUENCE LISTING

The material in the accompanying sequence listing is hereby incorporated by reference into this application. The accompanying sequence listing xml file, name ST26.xml, was created on and is kb.

### BACKGROUND OF THE INVENTION

### Background Information

Immunization remains one of the most effective public health measures to combat viral infections. Increased safety requirements for approval by the FDA, combined with the complexities associated with certain viral infections, have invigorated the search for new generation peptide/recombinant protein-based prophylactic vaccines. However, most recombinant proteins/peptides are not immunogenic and only induce weak immune responses when administered. Hence, recombinant protein/peptide vaccines often require an adjuvant to stimulate or enhance the immune response to antigens.

There are a wide variety of adjuvants approved or under study for recombinant protein-based vaccines with different immune modulatory properties. Many adjuvants promote strong antibody responses, e.g., alum, squalene or monophosphoryl lipid A, while others, e.g., CpG or other TLR 7, 8 or 9 agonists promote stronger Th1 CD4 and CD8 T cell responses. But even these distinctions are blurred somewhat because a single adjuvant can promote different responses through distinct pathways. For example, squalene-based adjuvants induce CD8 T cell responses through a pathway distinct from its antibody inducing property. Thus, it is now appreciated that different adjuvants evoke distinct immunological signatures that render them more or less effective for distinct diseases, e.g., tuberculosis vs influenza. Vaccines against respiratory viruses e.g., influenza, predominantly induce antibody responses and, for influenza, a neutralizing antibody titer is the primary correlate of immunity. However, natural infection also induces potent CD4 and CD8 T cell responses which are critical in forming long lasting immunity. CD8 T cell responses to internal proteins are also critical in situations where antigenic drift or shift prevents antibody recognition. Thus, it is now widely recognized that next generation universal vaccines against respiratory viral pathogens should induce strong neutralizing antibody as well as strong CD8 T cell immunity.

Lipid nanoparticles are some of the most promising delivery vehicles for eukaryotic cells and have been widely used since the late 1980s for the delivery of nucleic acids into cells including human gene therapy clinical trials. The novel mRNA vaccine technologies used to fight the COVID 19 pandemic also use lipid nanoparticles as delivery vehicles to deliver mRNA into cells. In recent years, cationic lipids have become attractive targets for delivering proteins or peptides for use in immunotherapy and vaccines. Importantly, cationic lipid mediated antigen uptake delivers proteins and peptides into the MHC class I and class II processing pathway. Mechanistically, cationic lipid nanoparticles efficiently bind negatively charged cell membranes in a receptor-independent manner and are rapidly internalized into endosomes in amounts exceeding receptor-mediated uptake. Once in endosomes, cationic lipids fuse with the endosomal membrane, delivering some of their contents to the cytoplasm. Thus, specific cationic lipids are ideally suited as non-viral vectors to deliver peptides, proteins, and inactivated whole viruses intracellularly into the MHC class I and II pathways. More recent studies investigating cationic lipids as delivery agents also identified that certain cationic lipids possess immune-stimulatory properties and can activate pathways essential for effective immune responses following vaccination. However, because cationic lipids differ greatly in their immunostimulatory properties and mechanisms of action, these characteristics are not universally exhibited by all cationic lipids.

The enantio-specific cationic lipid 1,2-dioleoyl-3-trimethylammonium-propane (R-DOTAP) has been shown to be particularly robust at inducing CD8 T cell responses to peptide-based vaccines. Further studies revealed that R-DOTAP promoted cellular uptake and cross-presentation of CD8 epitopes from long peptides and promoted the formation of polyfunctional CD8 T cells. R-DOTAP alone was shown to induce type I interferons in the draining LN and type I interferon was required for the R-DOTAP mediated induction of antigen specific CD8 T cells. While it is well established that R-DOTAP facilitates robust CD8 T cell responses to peptide-based vaccines, the ability of R-DOTAP to promote CD8 T cell responses to larger recombinant proteins is less clear. While R-DOTAP was also shown to induce robust antibody responses to the model antigen, OVA, there have been no studies on the ability of R-DOTAP to promote antibody responses to vaccine relevant recombinant proteins.

### SUMMARY OF THE INVENTION

The present invention is based on the seminal discovery that the use of a cationic lipid as an immunomodulator enhances immunity induced by influenza recombinant protein in vaccines compositions.

In one embodiment, the present invention provides a vaccine composition including one or more non-naturally occurring recombinant influenza antigens; and a cationic lipid.

In one aspect, the one or more non-naturally occurring recombinant influenza antigens include a computationally optimized broadly reactive influenza antigen (COBRA) hemagglutinins (HA). In another aspect, the one or more non-naturally occurring recombinant influenza antigens are recombinant H1N1 and/or H3N2 hemagglutinin influenza proteins. In one aspect, the one or more non-naturally occurring recombinant influenza antigens include an amino acid sequence with at least 80%, 85%90% or 95% sequence identity to any of SEQ ID NOs:3-22 and combinations thereof. In one aspect, the one or more non-naturally occurring recombinant influenza antigens include an amino acid sequence with at least 80%, 85%90% or 95% sequence identity to SEQ ID NO: 3 or SEQ ID NO:4. In another aspect, the one or more non-naturally occurring recombinant influenza antigens include an amino acid sequence of any of SEQ ID NOs:3-22 and combinations thereof. In another aspect, the one or more non-naturally occurring recombinant influenza antigens include an amino acid sequence of SEQ ID NO:3 or SEQ ID NO:4. In another aspect, the one or more non-naturally occurring recombinant influenza antigens include an amino acid sequence with at least 80%, 85%90% or 95% sequence identity to of SEQ ID NOs:3 and SEQ ID NO:4. In one aspect, the one or more non-naturally occurring recombinant influenza antigens include an amino acid sequence of SEQ ID NO: 3 and SEQ ID NO:4. In one aspect, the one or more non-naturally occurring recombinant influenza antigens include an amino acid sequence of SEQ ID NO: 3 and SEQ ID NO:4. In another aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In another aspect, the one or more non-naturally occurring recombinant influenza antigens are encapsulated in liposomes including cationic lipids. In another aspect, the one or more non-naturally occurring recombinant influenza antigens are mixed with preformed cationic lipid nanoparticles. In some aspects, the one or more non-naturally occurring recombinant influenza antigens and the preformed cationic lipid nanoparticles are mixed at a 1:1 ratio. In one aspect, the vaccine composition is a universal influenza vaccine.

In another embodiment, the invention provides a method of inducing an immune response against influenza viruses in a subject including administering to the subject a vaccine composition including: a) one or more non-naturally occurring recombinant influenza antigens; and b) a cationic lipid, thereby inducing an immune response against influenza viruses in a subject.

In one aspect, the immune response includes an induction of CD8⁺ effector T cells, CD4⁺ effector T cells and memory T cells. In another aspect, inducing CD8⁺ effector T cells and CD4⁺ effector T cells includes inducing proliferation of IFNγ and granzyme B producing CD8⁺ effector T cells, and/or the proliferation of IL-4 producing CD4⁺ effector T cells in the subject. In another aspect, inducing a humoral immune response includes inducing the production of IgG in the subject. In some aspects, IgG include IgG1 and IgG2a. In one aspect, inducing the immune response includes inducing the secretion of broadly neutralizing antibodies.

In an additional embodiment, the invention provides a method of preventing or treating an influenza infection in a subject including administering to the subject a vaccine composition including: a) one or more non-naturally occurring recombinant influenza antigens; and b) a cationic lipid, thereby preventing or treating an influenza infection in a subject.

In one embodiment, the invention provides a method of enhancing immunogenicity of an influenza vaccine in a subject including administering to the subject a vaccine composition including: a) one or more non-naturally occurring recombinant influenza antigens; and b) a cationic lipid, thereby enhancing immunogenicity of an influenza vaccine.

In one aspect, the influenza vaccine is an inactivated flu vaccine, an attenuated flu vaccine or a recombinant flu vaccine. In another aspect, the influenza vaccine is a monovalent vaccine, a bivalent vaccine, a trivalent vaccine or a quadrivalent vaccine.

In another embodiment, the invention provides a method of inducing secretion of broadly neutralizing antibodies against influenza viruses in a subject including administering to the subject a vaccine composition including: a) one or more non-naturally occurring recombinant influenza antigens; and b) a cationic lipid, thereby inducing secretion of broadly neutralizing antibodies against influenza viruses in a subject.

In a further embodiment, the invention provides a method of inducing a balanced Th1/Th2 immune response in a subject including administering to the subject a vaccine composition including: a) one or more non-naturally occurring recombinant influenza antigens; and b) a cationic lipid, thereby inducing secretion of broadly neutralizing antibodies against influenza viruses in a subject.

In one aspect, inducing a Th1 immune response includes inducing proliferation of IFNγ and granzyme B producing CD8+ effector T cells, and/or the proliferation of IL-4 producing CD4+ effector T cells in the subject. In some aspect, IFNγ producing CD8+ effector T cells are associated with production of IgG2a, and IL-4 producing CD4+ effector T cells are associated with production of IgG1.

In an additional embodiment, the invention provides a method of inducing a polyfunctional CD4+/CD8+ T cell response against influenza viruses in a subject including administering to the subject an influenza vaccine composition including: a) one or more non-naturally occurring recombinant influenza antigens; and b) a cationic lipid, thereby inducing a polyfunctional CD4+/CD8+ T cell response against influenza viruses in the subject.

In one aspect, inducing a polyfunctional CD4+/CD8+ T cell response includes inducing the secretion of two or more cytokines. In some aspects, the two or more cytokines are selected from the group consisting of IFNγ, granzyme B and IL-4.

### BRIEF DESCRIPTION OF THE DRAWINGS

FIGS. 1A-1B illustrate the antibody mediated immune response induced by recombinant influenza protein formulated with R-DOTAP. FIG. 1A is a graph showing total IgG antibody titer obtained with R-DOTAP preparations. FIG. 1B is a graph showing total IgG antibody titer obtained with Y2-COBRA preparations and sucrose.

FIGS. 2A-2C illustrate average particle size for R-DOTAP liposomal nanoparticles and R-DOTAP admixed with COBRA Y2 or COBRA-NG2 protein antigens. FIG. 2A is a graph illustrating particle size distribution. FIG. 2B is a transmission electron microscopy (TEM) photograph illustrating R-DOTAP nanoparticles. FIG. 2C is a TEM photograph illustrating R-DOTAP mixed with COBRA-Y2 and COBRA-NG2 mixtures resuspended in water.

FIGS. 3A-3D illustrate T cell responses to R-DOTAP formulations containing nucleoprotein and COBRA HA antigens FIG. 3A is a graph showing the number of antigen specific IFN-γ producing T cells in spleens in response to vaccines with influenza nucleoproteins.

FIG. 3B is a graph showing the number of antigen specific IFN-γ producing T cells in spleens in response to monovalent and bivalent vaccines with influenza recombinant COBRA sequences.

FIG. 3C is a graph illustrating specific T cell response to H1N1. FIG. 3D is a graph illustrating specific T cell response to H3N2.

FIGS. 4A-4E illustrate antibody mediated immune responses to COBRA proteins.

FIG. 4A a is a graph showing antigen specific antibody titers after vaccination with one or two doses. FIG. 4B is a graph comparing anti-Y2 antigen specific antibody titers 35 and 62 days after vaccination using sucrose, R-DOTAP or Addavax™ as the adjuvant. FIG. 4C is a graph comparing anti-NG2 antigen specific antibody titers 35 and 62 days after vaccination using sucrose, R-DOTAP or Addavax™ as the adjuvant. FIG. 4D is a graph comparing anti-Y2 antigen specific IgG1 and IgG2a antibody titers 35 and 62 days after vaccination using sucrose, R-DOTAP or Addavax™ as the adjuvant. FIG. 4E is a graph comparing anti-NG2 antigen specific IgG1 and IgG2a antibody titers 35 and 62 days after vaccination using sucrose, R-DOTAP or Addavax™ as the adjuvant.

FIGS. 5A-5B illustrate the ability of bivalent COBRA antigens formulated with R-DOTAP to induce Th1 and Th2 antibody responses. FIG. 5A is graph illustrating Th1 specific antibody titers. FIG. 5B is graph illustrating Th1 specific antibody titers.

FIGS. 6A-6C illustrate HAI titer on day 35 after vaccination with influenza virus alone or in combination with R-DOTAP nanoparticles. FIG. 6A is a graph illustrating HAI titers when the inactivated influenza vaccine, Fluzone® (2011-12 formulation) was a split virus hemagglutinin preparation derived from A/California/07/2009 X-179A (H1N1). FIG. 6B is a graph illustrating HAI titers when the inactivated influenza vaccine, Fluzone® (2011-12 formulation) was a split virus hemagglutinin preparation derived from A/Victoria/210/2009 X-187 (an A/Perth/16/2009-like virus) (H3N2). FIG. 6C is a graph illustrating HAI titers when the inactivated influenza vaccine, Fluzone® (2011-12 formulation) was a split virus hemagglutinin preparation derived from and B/Brisbane/60/2008.

FIGS. 7A-7D illustrate the effects of the presence of R-DOTAP in the vaccine formulation after vaccination and viral challenge. FIG. 7A is a graph illustrating body weight changes over time after vaccination and infection. FIG. 7B is a Kaplan Mayers graph illustrating survival post infection. FIG. 7C is a graph illustrating virus titers 3 days post infection. FIG. 7D is a graph illustrating virus titers 6 days post infection.

FIG. 8 is a schematic representation of the pre-immune ferret model.

FIGS. 9A-9C illustrate the results of H1N1 HAI response. FIG. 9A is a graph illustrating HAI titers in animals vaccinated with Y2+NG2 HA proteins. FIG. 9B is a graph illustrating HAI titers in animals vaccinated with Mich/15+Sing/16 HA proteins. FIG. 9C is a schematic representation showing when during the experiment timeline blood was collected to measure the data in FIGS. 9A and 9B.

FIGS. 10A-10C illustrate the results of H3N2 HAI response. FIG. 10A is a graph illustrating HAI titers in animals vaccinated with Y2+NG2 HA proteins. FIG. 10B is a graph illustrating HAI titers in animals vaccinated with Mich/15+Sing/16 HA proteins. FIG. 10C is a schematic representation showing when during the experiment timeline blood was collected to measure the data in FIGS. 10A and 10B.

FIG. 11A-11C illustrate further characterization of the response in animals vaccinated with Y2+NG2 HA proteins. FIG. 11A is a graph illustrating the body weight of the animals during the study. FIG. 11B is a graph illustrating D3 nasal wash viral titers. FIG. 11C is a schematic representation showing when during the experiment timeline nasal was performed to measure the data in FIG. 11B.

### DETAILED DESCRIPTION OF THE INVENTION

The present invention is based on the seminal discovery that that the use of a cationic lipid as an immunomodulator enhances immunity induced by influenza recombinant antigens in vaccine compositions.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

### Vaccines Compositions

In one embodiment, the present invention provides a vaccine composition including: one or more non-naturally occurring recombinant influenza antigens; and a cationic lipid.

As used herein the term composition is meant to include pharmaceutical compositions, which may also contain other therapeutic agents, and may be formulated, for example, by employing conventional pharmaceutically acceptable vehicles or diluents, as well as pharmaceutical additives of a type appropriate to the mode of desired administration (for example, excipients, preservatives, etc.) according to techniques known in the art of pharmaceutical formulation. In certain embodiments, the compositions disclosed herein are formulated with additional agents that promote entry into the desired cell or tissue. Such additional agents include micelles, liposomes, and dendrimers.

The term “pharmaceutically acceptable” refers to the fact that the carrier, diluent or excipient must be compatible with the other ingredients of the formulation and not deleterious to the recipient thereof. For example, the carrier, diluent, or excipient or composition thereof may be administered to a subject along with a conjugate of the invention without causing any undesirable biological effects or interacting in an undesirable manner with any of the other components of the pharmaceutical composition in which it is contained.

Pharmaceutical compositions including the peptides or compositions described herein may be administered by any suitable means, for example, parenterally, such as by subcutaneous, intravenous, intramuscular, intrathecal, or intracisternal injection or infusion techniques (e.g., as sterile injectable aqueous or non-aqueous solutions or suspensions) in dosage formulations containing non-toxic, pharmaceutically acceptable vehicles or diluents. Depending on the condition being treated, these pharmaceutical compositions may be formulated and administered systemically or locally. Techniques for formulation and administration are generally known in the art. Suitable routes may, for example, parenteral delivery, including intramuscular, subcutaneous, intramedullary, intrathecal, intraventricular, intravenous, or intraperitoneal. For injection, the pharmaceutical compositions of the invention may be formulated in aqueous solutions, preferably in physiologically compatible buffers such as water, Hanks' solution, Ringer's solution, or physiologically buffered saline.

The compositions of the invention are “vaccine compositions”, as used herein the term “vaccine” relates to a pharmaceutical preparation (pharmaceutical composition) or product that upon administration induces an immune response, in particular a cellular immune response, which recognizes and attacks a pathogen or a diseased cell such as a cancer cell. A vaccine may be used for the prevention or treatment of a disease.

The term “universal flu vaccine” concerns influenza vaccines in particular and is formulated to provide immune protection against at least two variants of an influenza virus. In one aspect, the vaccine compositions described herein are universal flu vaccines.

In one aspect, the vaccine compositions described herein provide immune protection against at least 2, 3, 4, 5, 6, 7, 8, 9, or 10 variants of an influenza virus. The vaccine compositions described herein provide immune protection against at least 3 variants of an influenza virus. the vaccine compositions described herein provide immune protection against at least 4 variants of an influenza virus. The vaccine compositions described herein provide immune protection against at least 5 variants of an influenza virus. The vaccine compositions described herein provide immune protection against at least 6 variants of an influenza virus. The vaccine compositions described herein provide immune protection against at least 7 variants of an influenza virus. The vaccine compositions described herein provide immune protection against at least 8 variants of an influenza virus. The vaccine compositions described herein provide immune protection against at least 9 variants of an influenza virus. The vaccine compositions described herein provide immune protection against at least 10 variants of an influenza virus.

In one aspect, examples of influenza variants include, but are not limited to H1N1, H3N2, H5N1, and H7N9. In one aspect, the vaccine compositions described herein provide immune protection against at least H1N1 and/or H3N2 hemagglutinin influenza variants.

The vaccine compositions described herein include one or more recombinant influenza antigens.

In one aspect, the one or more recombinant influenza antigens are recombinant H1N1 and/or H3N2 hemagglutinin influenza proteins.

The terms “peptide”, “polypeptide” and “protein” are used interchangeably herein and refer to any chain of at least two amino acids, linked by a covalent chemical bound. As used herein polypeptide can refer to the complete amino acid sequence coding for an entire protein or to a portion thereof. A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the coding sequence.

An “antigen” according to the invention covers any substance that will elicit an immune response. In particular, an “antigen” relates to any substance, preferably a peptide or protein, that reacts specifically with antibodies or T-lymphocytes (T cells). According to the present invention, the term “antigen” comprises any molecule which comprises at least one epitope. Preferably, an antigen in the context of the present invention is a molecule which, optionally after processing, induces an immune reaction. According to the present invention, any suitable antigen may be used, which is a candidate for an immune reaction, wherein the immune reaction is preferably a cellular immune reaction. In the context of the embodiments of the present invention, the antigen is preferably presented by a cell, preferably by an antigen presenting cell which includes a diseased cell, in particular a cancer cell, in the context of MHC molecules, which results in an immune reaction against the antigen. An antigen is preferably a product which corresponds to or is derived from a naturally occurring antigen. Such naturally occurring antigens include tumor antigens.

In another aspect, the one or more recombinant influenza antigens include computationally optimized broadly reactive influenza antigen (COBRA) hemagglutinins (HA). The COBRA HAs described herein are further described in detail in International Patent Application No. PCT/US2022/032799, U.S. Pat. No. 9,212,207 and the scientific publications Allen and Ross (“Bivalent H1 and H3 COBRA Recombinant Hemagglutinin Vaccines Elicit Seroprotectective Antibodies against H1N1 and H3N2 Influenza Viruses from 2009 to 2019”; J. Virology (2022); 96(7)) and Henson et al. (“R-DOTAP Cationic Lipid Nanoparticles Outperform Squalene-Based Adjuvant Systems in Elicitation of CD4 T Cells after Recombinant Influenza Hemagglutinin”; Viruses (2023): 15:538), which are incorporated herein by reference in its entirety.

As used herein, the “non-naturally occurring” peptide or antigen means a peptide or antigen that is not found in nature and that is comprised of one or more peptides or antigens, naturally occurring or non-naturally occurring, combined into a single peptide or antigen.

In one aspect, the one or more non-naturally occurring recombinant influenza antigens include an amino acid sequence with at least 80%, 85%90% or 95% sequence identity to any of SEQ ID NOs:3-22 and combinations thereof. In one aspect, the one or more non-naturally occurring recombinant influenza antigens include an amino acid sequence with at least 80%, 85% 90% or 95% sequence identity to SEQ ID NO: 3 or SEQ ID NO:4. In another aspect, the one or more non-naturally occurring recombinant influenza antigens include an amino acid sequence of any of SEQ ID NOs:3-22 and combinations thereof. In another aspect, the one or more non-naturally occurring recombinant influenza antigens include an amino acid sequence of SEQ ID NO:3 or SEQ ID NO:4. In another aspect, the one or more non-naturally occurring recombinant influenza antigens include an amino acid sequence with at least 80%, 85%90% or 95% sequence identity to of SEQ ID NOs:3 and SEQ ID NO:4. In one aspect, the one or more non-naturally occurring recombinant influenza antigens include an amino acid sequence of SEQ ID NO: 3 and SEQ ID NO:4. In one aspect, the one or more non-naturally occurring recombinant influenza antigens include an amino acid sequence of SEQ ID NO: 3 and SEQ ID NO:4.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 85%, at least 90%, at least 95%, at least 96%, at least 97%, at least 98%, at least 99%, at least 99.5%, or at least 99.9%. In general, an exact match indicates 100% identity over the length of the reference sequence.

Polypeptides and polynucleotides that are about 85, 86, 87, 88, 89, 90, 91, 92, 93, 94, 95, 96, 97, 98, 99 99.5% or more identical to polypeptides and polynucleotides described herein are embodied within the disclosure. For example, a polypeptide can have 80%, 85%, 90%, 91%, 92%, 93%, 94%, 95%, 96%, 97%, 98%, 99% or more identity to the sequences of the recombinant COBRA proteins described herein.

Variants of the disclosed sequences also include peptides, or full-length protein, that contain substitutions, deletions, or insertions into the protein backbone, that would still leave at least about 70% homology to the original protein over the corresponding portion. A yet greater degree of departure from homology is allowed if like-amino acids, i.e., conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

In one aspect, the recombinant influenza antigens have a sequence with at least 80% sequence identity with the sequence of SEQ ID NO: 3. In one aspect, the recombinant influenza antigens have a sequence with at least 85% sequence identity with the sequence of SEQ ID NO: 3. In one aspect, the recombinant influenza antigens have a sequence with at least 95% sequence identity with the sequence of SEQ ID NO: 3. In one aspect, the recombinant influenza antigens have a sequence with at least 96% sequence identity with the sequence of SEQ ID NO: 3. In one aspect, the recombinant influenza antigens have a sequence with at least 97% sequence identity with the sequence of SEQ ID NO: 3. In one aspect, the recombinant influenza antigens have a sequence with at least 98% sequence identity with the sequence of SEQ ID NO: 3. In one aspect, the recombinant influenza antigens have a sequence with at least 99% sequence identity with the sequence of SEQ ID NO: 3.

In one aspect, the recombinant influenza antigens have a sequence with at least 80% sequence identity with the sequence of SEQ ID NO: 4. In one aspect, the recombinant influenza antigens have a sequence with at least 85% sequence identity with the sequence of SEQ ID NO: 4. In one aspect, the recombinant influenza antigens have a sequence with at least 95% sequence identity with the sequence of SEQ ID NO: 4. In one aspect, the recombinant influenza antigens have a sequence with at least 96% sequence identity with the sequence of SEQ ID NO: 4. In one aspect, the recombinant influenza antigens have a sequence with at least 97% sequence identity with the sequence of SEQ ID NO: 4. In one aspect, the recombinant influenza antigens have a sequence with at least 98% sequence identity with the sequence of SEQ ID NO: 4. In one aspect, the recombinant influenza antigens have a sequence with at least 99% sequence identity with the sequence of SEQ ID NO: 4.

The vaccine compositions described herein include a cationic lipid.

Adjuvants are essential components of subunit vaccines added to enhance immune responses to antigens through immunomodulation. Very few adjuvants have been approved for human use by regulatory agencies due to safety concerns. Current subunit vaccine adjuvants approved for human use are very effective in promoting humoral immune responses but are less effective at promoting T cell immunity. In this study, a novel pure enantio-specific cationic lipid 1,2-dioleoyl-3-trimethylammonium-propane (R-DOTAP) was evaluated as an immunomodulator for subunit vaccines capable of inducing both humoral and cellular mediated immunity. Using recombinant protein antigens derived from computationally optimized broadly reactive influenza antigen (COBRA) proteins, it was demonstrated that R-DOTAP nanoparticles promoted strong cellular and antibody-mediated immune responses in both monovalent and bivalent vaccines. As further discussed below, R-DOTAP based vaccines induced antigen specific and polyfunctional CD8⁺ and CD4⁺ effector T cells and memory T cells, respectively. Antibody responses induced by R-DOTAP showed a balanced Th1/Th2 type immunity, neutralizing activity and protection of mice from challenge with live influenza viruses. R-DOTAP also facilitated significant dose sparing of the vaccine antigens. These studies demonstrate that R-DOTAP is an excellent immune stimulator for the production of next-generation subunit vaccines containing multiple recombinant proteins.

Adjuvants are often used to modify or augment the effects of a vaccine by stimulating the immune system to respond to the vaccine more vigorously, and thus providing increased immunity to a particular disease. Adjuvants accomplish this task by mimicking specific sets of evolutionarily conserved molecules, so called pathogen-associated molecular patterns, which include liposomes, lipopolysaccharide, molecular cages for antigens, components of bacterial cell walls, and endocytosed nucleic acids such as RNA, double-stranded RNA, single-stranded DNA, and unmethylated CpG dinucleotide-containing DNA. Because immune systems have evolved to recognize these specific antigenic moieties, the presence of an adjuvant in conjunction with the vaccine can greatly increase the innate immune response to the antigen by augmenting the activities of dendritic cells, lymphocytes, and macrophages by mimicking a natural infection.

The composition described herein can be formulated with a lipid nanoparticle as an adjuvant to enhance the presentation of the antigens to antigen presenting cells, and therefore to increase the immune response induce by the antigens.

In some aspects described herein, the adjuvant is a cationic lipid. As used herein, the term “cationic lipid” refers to any of a number of lipid species which carry a net positive charge at physiological pH or have a protonatable group and are positively charged at pH lower than the pKa.

Suitable cationic lipids according to the present disclosure include, but are not limited to: 3-β [4NIN, 8-diguanidino spermidine)-carbamoyl]cholesterol (BGSC); 3-β [N,N-diguanidinoethyl-aminoethane)-carbamoyl]cholesterol (BGTC); N,N.1N2N3Tetra-methyltetrapalmitylspermine (cellfectin); N-t-butyl-N′-tetradecyl-3-tetradecyl-aminopropion-amidine (CLONfectin); dimethyldioctadecyl ammonium bromide (DDAB); 1,2-dimyristyloxypropyl-3-dimethyl-hydroxy ethyl ammonium bromide (DMRIE); 2,3-dioleoyloxy-N-[2(sperminecarboxamido)ethyl]-N,N-dimethyl-1-p-ropanaminium trifluorocetate) (DOSPA); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3-dioleoyloxy-1,4-butane-diammonium iodide) (Tfx-50); N-1-(2,3-dioleoyloxy) propyl-N,N,N-trimethyl ammonium chloride (DOTMA) or other N-(N,N-1-dialkoxy)-alkyl-N,N,N-trisubstituted ammonium surfactants; 1,2dioleoyl-3-(4′-trimethylammonio) butanol-sn-glycerol (DOBT) or cholesteryl (4′trimethylammonia) butanoate (ChOTB) where the trimethylammonium group is connected via a butanol spacer arm to either the double chain (for DOTB) or cholesteryl group (for ChOTB); DORI (DL-1,2-dioleoyl-3-dimethylaminopropyl-.beta.-hydroxyethylammonium) or DORIE (DL-1,2-O-dioleoyl-3-dimethylaminopropyl-.beta.-hydroxyethylammoniu-m) (DORIE) or analogs thereof as disclosed in WO 93/03709; 1,2-dioleoyl-3-succinyl-sn-glycerol choline ester (DOSC); cholesteryl hemisuccinate ester (ChOSC); lipopolyamines such as dioctadecylamidoglycylspermine (DOGS) and dipalmitoyl phosphatidylethanolamylspermine (DPPES), cholesteryl-3β-carboxyl-amido-ethylenetrimethylammonium iodide, 1-dimethylamino-3-trimethylammonio-DL-2-propyl-cholesteryl carboxylate iodide, cholesteryl-3-O-carboxyamidoethyleneamine, cholesteryl-3-.beta.-oxysuccinamido-ethylenetrimethylammonium iodide, 1-dimethylamino-3-trimethylammonio-DL-2-propyl-cholesteryl-3-β-oxysu-ccinate iodide, 2-(2-trimethylammonio)-ethylmethylamino ethyl-cholesteryl-3-.Beta.-oxysuccinate iodide, 3-β-N-(N′,N′-dimethylaminoethane) carbamoyl cholesterol (DC-chol), and 3-β-N-(polyethyleneimine)-carbamoylcholesterol; O,O′-dimyristyl-N-lysyl aspartate (DMKE); 0,0′-dimyristyl-N-lysyl-glutamate (DMKD); 1,2-dimyristyloxypropyl-3-dimethyl-hydroxy ethyl ammonium bromide (DMRIE); 1,2-dilauroyl-sn-glycero-3-ethylphosphocholine (DLEPC); 1,2-dimyristoyl-sn-glycero-3-ethylphosphocholine (DMEPC); 1,2-dioleoyl-sn-glycero-3-ethylphosphocholine (DOEPC); 1,2-dipalmitoyl-sn-glycero-3-ethylphosphocholine (DPEPC); 1,2-distearoyl-sn-glycero-3-ethylphosphocholine (DSEPC); 1,2-dioleoyl-3-trimethylammonium propane (DOTAP); dioleoyl dimethylaminopropane (DODAP); 1,2-palmitoyl-3-trimethylammonium propane (DPTAP); 1,2-distearoyl-3-trimethylammonium propane (DSTAP), 1,2-myristoyl-3-trimethylammonium propane (DMTAP); and sodium dodecyl sulfate (SDS). Furthermore, structural variants and derivatives of the any of the described cationic lipids are also contemplated.

In some aspects, the cationic lipid is selected from the group consisting of DOTAP, DOTMA, DOEPC, and combinations thereof. In other aspects, the cationic lipid is DOTAP. In yet other aspects, the cationic lipid is DOTMA. In other aspects, the cationic lipid is DOEPC. In some aspects, the cationic lipid is purified.

In some embodiments, the cationic lipid is an enantiomer of a cationic lipid. The term “enantiomer” refers to a stereoisomer of a cationic lipid which is a non-superimposable mirror image of its counterpart stereoisomer, for example R and S enantiomers. In various examples, the enantiomer is R-DOTAP or S-DOTAP. In one example, the enantiomer is R-DOTAP. In another example, the enantiomer is S-DOTAP. In some aspects, the enantiomer is purified.

In one aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof.

In another aspect, the one or more recombinant influenza antigens are encapsulated in liposomes including cationic lipids. In another aspect, the one or more recombinant influenza antigens mixed with preformed cationic lipid nanoparticles.

In some aspects, the one or more recombinant protein antigens and the preformed cationic lipid nanoparticles are mixed at a 1:1 ratio.

In one aspect, the one or more recombinant influenza antigens are present as micelles separate from the cationic lipid nanoparticles.

### Methods of Use

In another embodiment, the invention provides a method of inducing an immune response against influenza viruses in a subject including administering to the subject a vaccine composition including: a) one or more non-naturally occurring recombinant influenza antigens; and b) a cationic lipid, thereby inducing an immune response against influenza viruses in a subject.

The term “immune response” refers to an integrated bodily response to an antigen and preferably refers to a cellular immune response or a cellular as well as a humoral immune response. The immune response may be protective/preventive/prophylactic and/or therapeutic.

The immune system is a system of biological structures and processes within an organism that protects against disease. This system is a diffuse, complex network of interacting cells, cell products, and cell-forming tissues that protects the body from pathogens and other foreign substances, destroys infected and malignant cells, and removes cellular debris: the system includes the thymus, spleen, lymph nodes and lymph tissue, stem cells, white blood cells, antibodies, and lymphokines. B cells or B lymphocytes are a type of lymphocyte in the humoral immunity of the adaptive immune system and are important for immune surveillance. T cells or T lymphocytes are a type of lymphocyte that plays a central role in cell-mediated immunity. There are two major subtypes of T cells: the killer T cell and the helper T cell. In addition, there are suppressor T cells which have a role in modulating immune response. Killer T cells only recognize antigens coupled to Class I MHC molecules, while helper T cells only recognize antigens coupled to Class II MHC molecules. These two mechanisms of antigen presentation reflect the different roles of the two types of T cell. A third minor subtype are the γδ T cells that recognize intact antigens that are not bound to MHC receptors. In contrast, the B cell antigen-specific receptor is an antibody molecule on the B cell surface and recognizes whole pathogens without any need for antigen processing. Each lineage of B cell expresses a different antibody, so the complete set of B cell antigen receptors represent all the antibodies that the body can manufacture.

A “cellular immune response”, a “cellular response”, a “cellular response against an antigen” or a similar term is meant to include a cellular response directed to cells characterized by presentation of an antigen with class I or class II MHC. The cellular response relates to cells called T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells. In preferred embodiments, the present invention involves the stimulation of an anti-tumor CTL response against tumor cells expressing one or more tumor expressed antigens and preferably presenting such tumor expressed antigens with class I MHC.

The terms “immunoreactive cell” “immune cells” or “immune effector cells” in the context of the present invention relate to a cell which exerts effector functions during an immune reaction. An “immunoreactive cell” preferably is capable of binding an antigen or a cell characterized by presentation of an antigen, or an antigen peptide derived from an antigen and mediating an immune response. For example, such cells secrete cytokines and/or chemokines, secrete antibodies, recognize cancerous cells, and optionally eliminate such cells. For example, immunoreactive cells comprise T cells (cytotoxic T cells, helper T cells, tumor infiltrating T cells), B cells, natural killer cells, neutrophils, macrophages, and dendritic cells.

“Inducing an immune response” may mean that there was no immune response against a particular antigen before induction, but it may also mean that there was a certain level of immune response against a particular antigen before induction and after induction said immune response is enhanced. Thus, “inducing an immune response” also includes “enhancing an immune response”. Preferably, after inducing an immune response in a subject, said subject is protected from developing a disease such as the flu or the disease condition is ameliorated by inducing an immune response. For example, an immune response against an influenza antigen may be induced in a subject at risk of being infected by an influenza virus. Inducing an immune response in this case may mean that the disease condition of the subject is ameliorated, or that the subject does not develop the flu.

In one aspect, the immune response includes an induction of CD8⁺ effector T cells, CD4⁺ effector T cells and memory T cells.

In one aspect, inducing CD8+ and CD4+ effector T cells includes inducing proliferation of IFNγ and granzyme B producing CD8+ effector T cells, and/or the proliferation of IL-4 producing CD4+ effector T cells in the subject.

In another aspect, inducing a humoral immune response includes inducing the production of IgG in the subject. In some aspects, IgG include IgG1 and IgG2a.

In one aspect, inducing the immune response includes inducing the secretion of broadly neutralizing antibodies.

In one aspect, the one or more non-naturally occurring recombinant influenza antigens include a computationally optimized broadly reactive influenza antigen (COBRA) hemagglutinins (HA). In another aspect, the one or more non-naturally occurring recombinant influenza antigens are recombinant H1N1 and/or H3N2 hemagglutinin influenza proteins. In one aspect, the one or more non-naturally occurring recombinant influenza antigens include an amino acid sequence with at least 80%, 85%90% or 95% sequence identity to any of SEQ ID NOs:3-22 and combinations thereof. In one aspect, the one or more non-naturally occurring recombinant influenza antigens include an amino acid sequence with at least 80%, 85%90% or 95% sequence identity to SEQ ID NO: 3 or SEQ ID NO:4. In another aspect, the one or more non-naturally occurring recombinant influenza antigens include an amino acid sequence of any of SEQ ID NOs:3-22 and combinations thereof. In another aspect, the one or more non-naturally occurring recombinant influenza antigens include an amino acid sequence of SEQ ID NO:3 or SEQ ID NO:4. In another aspect, the one or more non-naturally occurring recombinant influenza antigens include an amino acid sequence with at least 80%, 85%90% or 95% sequence identity to of SEQ ID NOs:3 and SEQ ID NO:4. In one aspect, the one or more non-naturally occurring recombinant influenza antigens include an amino acid sequence of SEQ ID NO: 3 and SEQ ID NO:4. In one aspect, the one or more non-naturally occurring recombinant influenza antigens include an amino acid sequence of SEQ ID NO: 3 and SEQ ID NO:4.

In another aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In another aspect, the one or more recombinant influenza antigens are encapsulated in liposomes including cationic lipids. In another aspect, the one or more recombinant influenza antigens are mixed with preformed cationic lipid nanoparticles. In some aspects, the one or more recombinant influenza antigens and the preformed cationic lipid nanoparticles are mixed at a 1:1 ratio. In one aspect, the one or more recombinant influenza antigens are present as micelles separate from the cationic lipid nanoparticles.

In an additional embodiment, a method of preventing or treating an influenza infection in a subject including administering to the subject a vaccine composition including: a) one or more non-naturally occurring recombinant influenza antigens; and b) a cationic lipid, thereby preventing or treating an influenza infection in a subject.

In one aspect, the one or more non-naturally occurring recombinant influenza antigens include a computationally optimized broadly reactive influenza antigen (COBRA) hemagglutinins (HA). In another aspect, the one or more non-naturally occurring recombinant influenza antigens are recombinant H1N1 and/or H3N2 hemagglutinin influenza proteins. In one aspect, the one or more non-naturally occurring recombinant influenza antigens include an amino acid sequence with at least 80%, 85%90% or 95% sequence identity to any of SEQ ID NOs:3-22 and combinations thereof. In one aspect, the one or more non-naturally occurring recombinant influenza antigens include an amino acid sequence with at least 80%, 85%90% or 95% sequence identity to SEQ ID NO: 3 or SEQ ID NO:4. In another aspect, the one or more non-naturally occurring recombinant influenza antigens include an amino acid sequence of any of SEQ ID NOs:3-22 and combinations thereof. In another aspect, the one or more non-naturally occurring recombinant influenza antigens include an amino acid sequence of SEQ ID NO:3 or SEQ ID NO:4. In another aspect, the one or more non-naturally occurring recombinant influenza antigens include an amino acid sequence with at least 80%, 85%90% or 95% sequence identity to of SEQ ID NOs:3 and SEQ ID NO:4. In one aspect, the one or more non-naturally occurring recombinant influenza antigens include an amino acid sequence of SEQ ID NO: 3 and SEQ ID NO:4. In one aspect, the one or more non-naturally occurring recombinant influenza antigens include an amino acid sequence of SEQ ID NO: 3 and SEQ ID NO:4.

In another aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In another aspect, the one or more recombinant influenza antigens are encapsulated in liposomes including cationic lipids. In another aspect, the one or more recombinant influenza antigens are mixed with preformed cationic lipid nanoparticles. In some aspects, the one or more recombinant influenza antigens and the preformed cationic lipid nanoparticles are mixed at a 1:1 ratio. In one aspect, the one or more recombinant influenza antigens are present as micelles separate from the cationic lipid nanoparticles.

In one embodiment, the invention provides a method of enhancing immunogenicity of an influenza vaccine in a subject including administering to the subject a vaccine composition including: a) one or more non-naturally occurring recombinant influenza antigens; and b) a cationic lipid, thereby enhancing immunogenicity of an influenza vaccine.

In some aspects administration can be in combination with one or more additional therapeutic agents. The phrases “combination therapy”, “combined with” and the like refer to the use of more than one medication or treatment simultaneously to increase the response. The cationic lipid of the present invention might for example be used in combination with existing influenza vaccine to increase the immune response generated by the influenza vaccine alone. The cationic lipid can be administered prior to, simultaneously with, or following administration of the influenza vaccine.

By “enhancing immunogenicity”, it is meant that the immunogenicity of the influenza vaccine is greater when it is administered in combination with the cationic lipid of the present invention, as compared to the immunogenicity induced by the influenza vaccine administered alone (e.g., in the absence of the administration of the cationic lipid as an immunomodulator).

In one aspect, the influenza vaccine is an inactivated flu vaccine, an attenuated flu vaccine or a recombinant flu vaccine.

Vaccines typically contain attenuated, inactivated or dead organisms or purified products derived from them. There are several types of vaccines in use, representing different strategies used to try to reduce the risk of illness while retaining the ability to induce a beneficial immune response. Influenza vaccines are usually “attenuated”, “inactivated” or “subunit” (e.g., recombinant).

Live, attenuated microorganisms, such as active viruses that have been cultivated under conditions that disable their virulent properties, or that use closely related but less dangerous organisms to produce a broad immune response constitute attenuated vaccines. Although most attenuated vaccines are viral, some are bacterial in nature.

Inactivated, but previously virulent, micro-organisms that have been destroyed with chemicals, heat, or radiation-“ghosts”, with intact but empty bacterial cell envelopes constitute inactivated vaccines. They are considered an intermediate phase between the inactivated and attenuated vaccines. Examples include IPV (polio vaccine), hepatitis A vaccine, rabies vaccine and most influenza vaccines.

Rather than introducing an inactivated or attenuated micro-organism to an immune system (which would constitute a “whole-agent” vaccine), a subunit vaccine uses a fragment of it to create an immune response. Only one protein of the virus (previously extracted from the blood serum of chronically infected patients but now produced by recombination of the viral genes into yeast), for example a surface protein is recombinantly produced and used for the vaccine. The hemagglutinin and neuraminidase subunits of the influenza virus are examples or subunit proteins used in recombinant influenza vaccines.

In another aspect, the influenza vaccine is a monovalent vaccine, a bivalent vaccine, a trivalent vaccine or a quadrivalent vaccine.

In one aspect, the one or more non-naturally occurring recombinant influenza antigens include a computationally optimized broadly reactive influenza antigen (COBRA) hemagglutinins (HA). In another aspect, the one or more non-naturally occurring recombinant influenza antigens are recombinant H1N1 and/or H3N2 hemagglutinin influenza proteins. In one aspect, the one or more non-naturally occurring recombinant influenza antigens include an amino acid sequence with at least 80%, 85%90% or 95% sequence identity to any of SEQ ID NOs:3-22 and combinations thereof. In one aspect, the one or more non-naturally occurring recombinant influenza antigens include an amino acid sequence with at least 80%, 85%90% or 95% sequence identity to SEQ ID NO: 3 or SEQ ID NO:4. In another aspect, the one or more non-naturally occurring recombinant influenza antigens include an amino acid sequence of any of SEQ ID NOs:3-22 and combinations thereof. In another aspect, the one or more non-naturally occurring recombinant influenza antigens include an amino acid sequence of SEQ ID NO:3 or SEQ ID NO:4. In another aspect, the one or more non-naturally occurring recombinant influenza antigens include an amino acid sequence with at least 80%, 85%90% or 95% sequence identity to of SEQ ID NOs:3 and SEQ ID NO:4. In one aspect, the one or more non-naturally occurring recombinant influenza antigens include an amino acid sequence of SEQ ID NO: 3 and SEQ ID NO:4. In one aspect, the one or more non-naturally occurring recombinant influenza antigens include an amino acid sequence of SEQ ID NO: 3 and SEQ ID NO:4.

In another aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In another aspect, the one or more recombinant influenza antigens are encapsulated in liposomes including cationic lipids. In another aspect, the one or more recombinant influenza antigens are mixed with preformed cationic lipid nanoparticles. In some aspects, the one or more recombinant influenza antigens and the preformed cationic lipid nanoparticles are mixed at a 1:1 ratio. In one aspect, the one or more recombinant influenza antigens are present as micelles separate from the cationic lipid nanoparticles.

Examples of influenza vaccines include, but are not limited to Afluria Quadrivalent, Fluarix Quadrivalent, FluLaval Quadrivalent, Fluzone Quadrivalent, Flucelvax Quadrivalent, Fluzone High-Dose Quadrivalent, Fluad Quadrivalent, Flublok Quadrivalent, FluMist Quadrivalent and Fluzone®.

In another embodiment, the invention provides a method of inducing secretion of broadly neutralizing antibodies against influenza viruses in a subject including administering to the subject a vaccine composition including: a) one or more non-naturally occurring recombinant influenza antigens; and b) a cationic lipid, thereby inducing secretion of broadly neutralizing antibodies against influenza viruses in a subject.

Development of a universal influenza vaccine that can provide heterosubtypic immunity and protection against multiple clades has been a long-term goal to protect against Influenza infections. There are several strategies under investigation to achieve this goal, including the use of antigens from multiple subtypes to increase the coverage, targeting multiple proteins (e.g., inclusion of HA and non-HA proteins) as antigens, target conserved antigenic regions of influenza antigens, use of chimeric proteins containing stem and stalk HA from different subtypes, and use of consensuses based approaches such as COBRA sequences comprising multiple known mutations in the hemagglutinin or neuraminidase. In addition, vaccine technologies that induce both CD8 T cell and antibody-mediated immunity are also actively being sought to achieve heterosubtypic protection against influenza. As described herein, using a prototype vaccine based on the R-DOTAP platform and COBRA H1N1 and H3N2 derived HA or nucleoprotein antigen, that such a vaccine is capable of inducing antigen specific T cell responses, neutralizing antibodies against multiple clades of H1N1, and H3N2 strains, and protected mice against challenge with a lethal H1N1 strain. Hence, vaccine formulations containing R-DOTAP and COBRA HA, NA sequences, and nucleoprotein from influenza presents strong potential to further the goal of developing a safe and effective universal influenza vaccine.

The term “broadly neutralizing antibodies” or “bNAbs” as used herein is meant to refer to neutralizing antibodies which neutralize multiple influenza viral strains. bNAbs are unique in that they target conserved epitopes of the virus, meaning the virus may mutate, but the targeted epitopes will still exist. In contrast, non-bNAbs are specific for individual viral strains with unique epitopes.

In one aspect, the one or more non-naturally occurring recombinant influenza antigens include a computationally optimized broadly reactive influenza antigen (COBRA) hemagglutinins (HA). In another aspect, the one or more non-naturally occurring recombinant influenza antigens are recombinant H1N1 and/or H3N2 hemagglutinin influenza proteins. In one aspect, the one or more non-naturally occurring recombinant influenza antigens include an amino acid sequence with at least 80%, 85%90% or 95% sequence identity to any of SEQ ID NOs:3-22 and combinations thereof. In one aspect, the one or more non-naturally occurring recombinant influenza antigens include an amino acid sequence with at least 80%, 85%90% or 95% sequence identity to SEQ ID NO: 3 or SEQ ID NO:4. In another aspect, the one or more non-naturally occurring recombinant influenza antigens include an amino acid sequence of any of SEQ ID NOs:3-22 and combinations thereof. In another aspect, the one or more non-naturally occurring recombinant influenza antigens include an amino acid sequence of SEQ ID NO:3 or SEQ ID NO:4. In another aspect, the one or more non-naturally occurring recombinant influenza antigens include an amino acid sequence with at least 80%, 85%90% or 95% sequence identity to of SEQ ID NOs:3 and SEQ ID NO:4. In one aspect, the one or more non-naturally occurring recombinant influenza antigens include an amino acid sequence of SEQ ID NO: 3 and SEQ ID NO:4. In one aspect, the one or more non-naturally occurring recombinant influenza antigens include an amino acid sequence of SEQ ID NO: 3 and SEQ ID NO:4.

In another aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In another aspect, the one or more recombinant influenza antigens are encapsulated in liposomes including cationic lipids. In another aspect, the one or more recombinant influenza antigens are mixed with preformed cationic lipid nanoparticles. In some aspects, the one or more recombinant influenza antigens and the preformed cationic lipid nanoparticles are mixed at a 1:1 ratio. In one aspect, the one or more recombinant influenza antigens are present as micelles separate from the cationic lipid nanoparticles.

In a further embodiment, the invention provides a method of inducing a balanced Th1/Th2 immune response in a subject including administering to the subject a vaccine composition including: a) one or more non-naturally recombinant influenza antigens; and b) a cationic lipid, thereby inducing secretion of broadly neutralizing antibodies against influenza viruses in a subject.

Adjuvants can be broadly categorized into Th1, Th2, Th17, and mixed Th1/Th2 or Th1/Th17 types based on the cytokines and antibody subclasses induced by the vaccine. Th1 type adjuvants for example show skewing towards IFN-γ production and IgG2a/c antibody subtypes in mouse vaccinations. Th2 adjuvants stimulate more IL-4 production resulting in skewing towards IgG1 antibody subtypes in mice.

In one aspect, inducing a Th1 immune response includes inducing proliferation of IFNγ and granzyme B producing CD8+ effector T cells, and/or the proliferation of IL-4 producing CD4+ effector T cells in the subject.

In some aspects, IFNγ producing CD8+ effector T cells are associated with production of IgG2a, and IL-4 producing CD4+ effector T cells are associated with production of IgG1.

In one aspect, administering the vaccine composition to the subject includes subcutaneous administration or intramuscular administration.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual, buccal, rectal, vaginal, nasal, ocular administrations, as well infusion, inhalation, and nebulization. Preferably, the vaccine compositions described herein are administered through subcutaneous or intramuscular administration.

In another aspect, administering the vaccine composition includes administering two doses of the vaccines, and optionally administering a booster.

In one aspect, the one or more non-naturally recombinant influenza antigens include a computationally optimized broadly reactive influenza antigen (COBRA) hemagglutinins (HA). In another aspect, the one or more non-naturally recombinant influenza antigens are recombinant H1N1 and/or H3N2 hemagglutinin influenza proteins. In one aspect, the one or more non-naturally occurring recombinant influenza antigens include an amino acid sequence with at least 80%, 85% 90% or 95% sequence identity to any of SEQ ID NOs:3-22 and combinations thereof. In one aspect, the one or more non-naturally occurring recombinant influenza antigens include an amino acid sequence with at least 80%, 85%90% or 95% sequence identity to SEQ ID NO: 3 or SEQ ID NO:4. In another aspect, the one or more non-naturally occurring recombinant influenza antigens include an amino acid sequence of any of SEQ ID NOs:3-22 and combinations thereof. In another aspect, the one or more non-naturally occurring recombinant influenza antigens include an amino acid sequence of SEQ ID NO:3 or SEQ ID NO:4. In another aspect, the one or more non-naturally occurring recombinant influenza antigens include an amino acid sequence with at least 80%, 85% 90% or 95% sequence identity to of SEQ ID NOs:3 and SEQ ID NO:4. In one aspect, the one or more non-naturally occurring recombinant influenza antigens include an amino acid sequence of SEQ ID NO: 3 and SEQ ID NO:4. In one aspect, the one or more non-naturally occurring recombinant influenza antigens include an amino acid sequence of SEQ ID NO: 3 and SEQ ID NO:4.

In another aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In another aspect, the one or more recombinant influenza antigens are encapsulated in liposomes including cationic lipids. In another aspect, the one or more recombinant influenza antigens are mixed with preformed cationic lipid nanoparticles. In some aspects, the one or more recombinant influenza antigens and the preformed cationic lipid nanoparticles are mixed at a 1:1 ratio. In one aspect, the one or more recombinant influenza antigens are present as micelles separate from the cationic lipid nanoparticles.

In an additional embodiment, the invention provides a method of inducing a polyfunctional CD4+/CD8+ T cell response against influenza viruses in a subject including administering to the subject an influenza vaccine composition including: a) one or more non-naturally occurring recombinant influenza antigens; and b) a cationic lipid, thereby inducing a polyfunctional CD4+/CD8+ T cell response against influenza viruses in the subject.

In one aspect, inducing a polyfunctional CD4+/CD8+ T cell response includes inducing the secretion of two or more cytokines. In some aspects, the two or more cytokines are selected from the group consisting of IFNγ, granzyme B and IL-4.

In one aspect, the one or more non-naturally occurring recombinant influenza antigens include a computationally optimized broadly reactive influenza antigen (COBRA) hemagglutinins (HA). In another aspect, the one or more non-naturally occurring recombinant influenza antigens are recombinant H1N1 and/or H3N2 hemagglutinin influenza proteins. In one aspect, the one or more non-naturally occurring recombinant influenza antigens include an amino acid sequence with at least 80%, 85%90% or 95% sequence identity to any of SEQ ID NOs:3-22 and combinations thereof. In one aspect, the one or more non-naturally occurring recombinant influenza antigens include an amino acid sequence with at least 80%, 85%90% or 95% sequence identity to SEQ ID NO: 3 or SEQ ID NO:4. In another aspect, the one or more non-naturally occurring recombinant influenza antigens include an amino acid sequence of any of SEQ ID NOs:3-22 and combinations thereof. In another aspect, the one or more non-naturally occurring recombinant influenza antigens include an amino acid sequence of SEQ ID NO:3 or SEQ ID NO:4. In another aspect, the one or more non-naturally occurring recombinant influenza antigens include an amino acid sequence with at least 80%, 85%90% or 95% sequence identity to of SEQ ID NOs:3 and SEQ ID NO:4. In one aspect, the one or more non-naturally occurring recombinant influenza antigens include an amino acid sequence of SEQ ID NO: 3 and SEQ ID NO:4. In one aspect, the one or more non-naturally occurring recombinant influenza antigens include an amino acid sequence of SEQ ID NO: 3 and SEQ ID NO:4.

In another aspect, the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof. In one aspect, the cationic lipid is R-DOTAP. In another aspect, the one or more recombinant influenza antigens are encapsulated in liposomes including cationic lipids. In another aspect, the one or more recombinant influenza antigens are mixed with preformed cationic lipid nanoparticles. In some aspects, the one or more recombinant influenza antigens and the preformed cationic lipid nanoparticles are mixed at a 1:1 ratio. In one aspect, the one or more recombinant influenza antigens are present as micelles separate from the cationic lipid nanoparticles.

Presented below are examples discussing vaccines combination including influenza recombinant antigens and a cationic lipid contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLES

### Example 1

### Materials & Methods

Animals and viruses: Six to twenty-week-old C57BL/6J mice (B6 mice), BALB/cJ, K18-hACE2 mice (B6.Cg-Tg(K18-ACE2)2Prlmn/J), and DBA/2J, mice were obtained from Jackson Laboratories. All animals were housed in specific-pathogen-free conditions at the Division of Laboratory Animal Resources (DLAR), University of Kentucky Medical Center, or in the animal research center at the University of Georgia. All animal protocols were reviewed and approved by the University of Kentucky (2019-3226) or The University of Georgia Institutional Animal Care and Use Committee following the National Institutes of Health Animal care guidelines (A2020 02-024-Y1-A5, A2018 06-018-Y3-A16). All studies were carried out in compliance with ARRIVE guidelines.

For influenza challenge studies (A/Brisbane/02/2018(Bris/18) was used. For HAI assays (A/California/07/2009 (Cal/09), A/Guangdong-Maonan/SWL1536/2019(GD19), A/Singapore/IFNIMH-16-00192016 (Singapore/16), A/Hong Kong/4801/2014 (Hong Kong/14), A/Victoria/210/2009 X-187 (an A/Perth/16/2009-like virus) (H3N2), and B/Brisbane/60/2008 were used. Influenza virus were obtained from International influenza Resources (IRR).

### Reagents and Antibodies:

cGMP grade R-DOTAP (1,2-dioleoyl-3-trimethyl ammonium-propane) was provided by Merck & Cie. Evonik produced cGMP grade R-DOTAP liposomal nanoparticles according to protocols described previously.

Influenza COBRA antigens: COBRA HA-Y2 (COBRA-Y2) (H1N1), and COBRA-HA-NG2 (COBRA-NG2) (H3N2), were synthesized at the University of Georgia Center for Vaccines and Immunology Research center. Influenza nucleoprotein from A/Puerto Rico/8/34/Mount Sinai) was obtained from Sino biologicals US Inc. Fluzone® vaccine formulation was obtained from the University of Kentucky HealthCare pharmacy.

Overlapping peptide pools from Influenza A/New York/383/2005 (H3N2) hemagglutinin protein and overlapping peptide pools from Influenza A/California/07/2009 (H1N1) pdm09 were obtained from BEI Resources.

Fluorochrome conjugated mouse monoclonal anti-mouse CD3 (Clone: 145-2c11), CD4 (Clone: GK1.5), CD8 (Clone: YTS165.7.7), CD44 (clone: IM7), CD62L (clone: MED-14), IFNγ (Clone: XMG1.2), TNFα (Clone: MP6-XT22), IL-2 (Clone: JES6.5H4), were purchased from BioLegend.

### Preparation of R-DOTAP Nanoparticles and Vaccine Formulations:

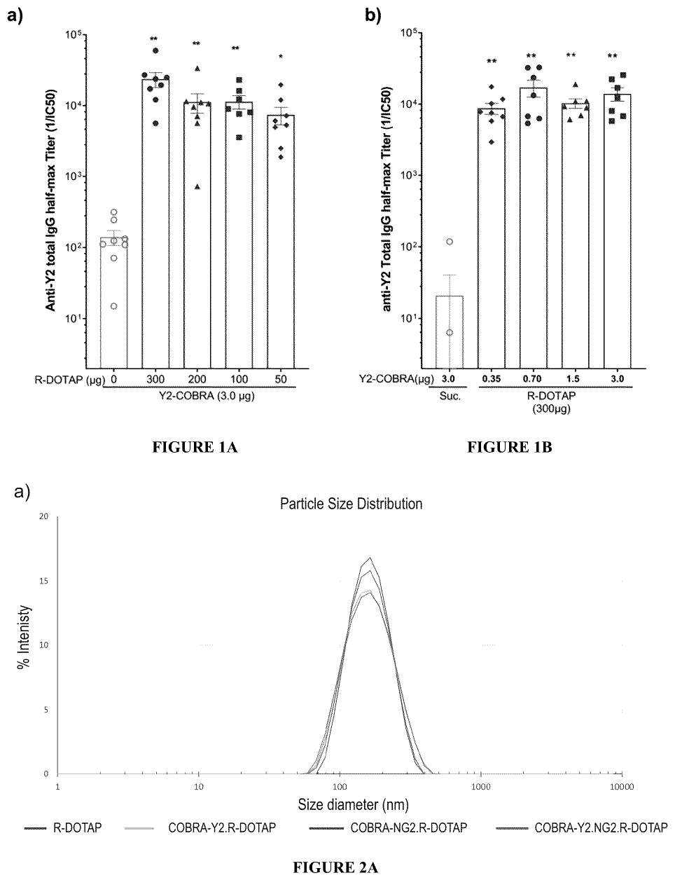

cGMP grade R-DOTAP liposomal nanoparticles were produced by Evonik using thin film hydration method according to protocols described previously. Briefly, R-DOTAP thin films were formed by dissolving the lipid in 1:1 chloroform and methanol mixture in a round bottom flask. Organic solvents were then evaporated using steady stream of dry nitrogen gas followed by overnight vacuum desiccation. The dried R-DOTAP film is then hydrated by incubating films in water for 12 hr. The lipid suspensions were then sonicated for 10 minutes and extruded sequentially using 400, 200, and 100 nm polycarbonate membrane filters to obtain uniform sized liposomal nanoparticles (FIGS. 1A-1B). Groups of BALB/cJ (n=6-8) mice were immunized on day 0 and day 21 with two doses of monovalent COBRA-Y2 formulated with R-DOTAP nanoparticles or sucrose buffer (sucrose). Serum samples obtained from vaccinated mice on day 35 (14 days after the second dose) were measured for anti-COBRA-Y2 total IgG antibody titer. Data represents (a-b) mean±SEM of half-max titers from each mouse. Comparisons between sucrose alone or R-DOTAP groups was performed using Student's t-test (unpaired-two tailed) ** P≤0.05. The nanoparticles were then diluted in 280 mM Sucrose buffer and were stored at −80° C. until use. For making vaccine formulations, concentrated antigens dissolved in PBS buffer was diluted to desired concentration in 280 mM sucrose. Prior to vaccination, the vaccine components were brought to ambient temperature and antigen component is then mixed 1:1 ratio with the R-DOTAP nanoparticle using a pipette to form a uniform suspension. For subcutaneous vaccine delivery, 100 μl were used for each dose; for intramuscular delivery, 50 μl were used for each dose.

### Physical Characterization of Vaccine Formulation:

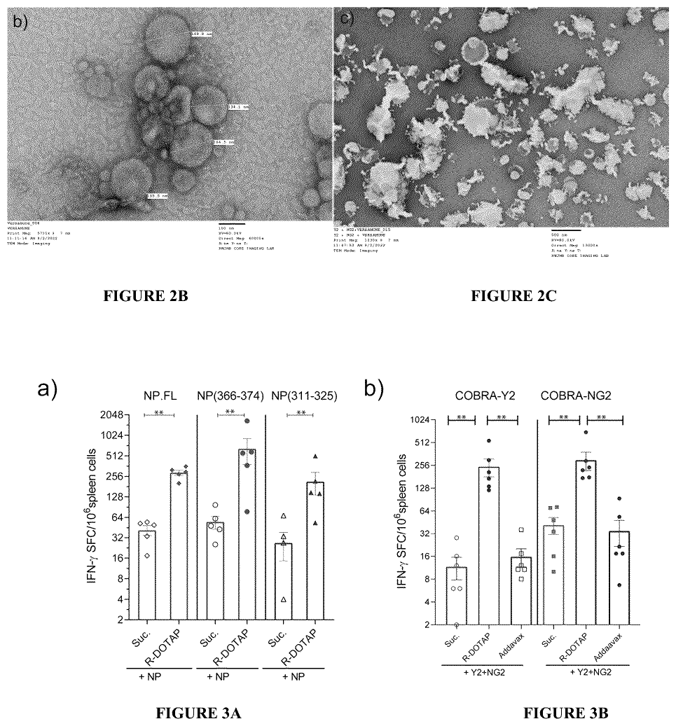

Particle size, polydispersity and zeta potential of R-DOTAP liposomes and vaccine formulations were measured at 23° C. using Zeasizer nano equipped with a 4 mW 632.8 nm laser set at 90° angle. Dynamic light scattering was used to determine the fluctuations in dispersed light intensity. Distribution analysis and cumulants analysis to measure Z average and polydispersity was performed according to instructions and using apparatus software. A representative particle size distribution is shown in FIGS. 2A-2C. Average particle size, polydispersity and zeta potential measurements are shown in Table 1.

### Enzyme-Linked Immunosorbent Assay (ELISA):

Blood was collected into B.D. Microtainer® serum separator tubes through tail vein bleed or cardiac puncture of euthanized mice. Isolated serum samples were stored at −80° C. until analysis. For antibody titer measurement, 96 well plates were coated overnight at 4° C. with 50 μl/well recombinant RBD protein, COBRA-NG2, or COBRA-Y2 protein at 2 μg/ml concentration. After antigen coating, the wells were blocked for 1-2 hr at room temperature with 200 μl/well of PBS-T buffer containing 3% non-fat dry milk and 0.1% tween-20. Following blocking, buffer was replaced with serum samples diluted in PBS-T buffer containing 1% non-fat dry milk. After 2 hr incubation at R.T., the serum was removed, and wells were washed four times with PBS-T buffer. For detecting protein antibodies, wells were incubated for 1 hr with 100 μl of PBS-T buffer containing 1% non-fat dry milk and HRP-conjugated anti-mouse IgG (1:5000) (cat #115-035-003 Jackson ImmunoResearch), anti-mouse IgG1 (1:5000) (cat #115-035-205; Jackson Immuno Research), anti-mouse IgG2c (1:5000) (Cat #115-035-206; Jackson ImmunoResearch) or anti-mouse IgG2a (1:5000) (Cat #115-035-206; Jackson ImmunoResearch). Wells were then washed four times, and 100 μl of SIGMAFAST OPD (o-phenylenediamine dihydrochloride) chromogenic substrate was added to each well. After 10 minutes of incubation, the reaction was stopped by adding 50 μl of 3M HCL, and absorbance measured at 490 nm (OD490) using a spectraMax M5 microplate reader.

### Enzyme-Linked Immunospot Assay (ELISpot):

2.5×10⁵ processed spleen cells were stimulated for 18-24 hr at 37° C. with T cell epitope peptides of interest or recombinant proteins, or no peptides (control) in a 96 well plate pre-coated with the mouse IFNγ or IL-4 capture antibody (Mabtech). After stimulation, wells were washed with PBS and incubated with biotin-conjugated anti-IFNγ or IL-4 antibody followed by streptavidin-HRP antibody. To visualize the antigen specific IFNγ or IL-4 producing cells, wells were incubated for 6 minutes with TMB substrate, washed with water, and air dried. Spots were scanned and counted using CTL ImmunoSpot Analyzer and ImmunoSpot Ver.6 software. Spot counts were summarized as median values from triplicate samples. Each sample had unstimulated and PMA/Ionomycin control wells to detect background or as a positive control.

### Intracellular Cytokine and Cell Surface Staining:

For intracellular protein analysis, single cell suspensions of spleen cells were stimulated with indicated stimulatory antigenic peptide for 6 hr at 37° C. in cRPMI media supplemented with purified anti-mouse CD28 (2 μg/ml), protein transport inhibitor Brefeldin A (5 μg/ml) and monensin (2.0 μM). Following stimulation, cells were washed with FACS buffer and stained with fluorochrome conjugated anti-mouse CD3, CD4, CD8, CD44, and CD62L antibodies. The cells were then washed, fixed and permeabilized using fixation/permeabilization kit and stained with fluorochrome conjugated anti-mouse IFNγ, TNFα, and IL-2. Cells were washed with FACS buffer after intracellular staining and analyzed immediately using flow cytometry.

### Mouse Vaccination:

Mice were anesthetized using isoflurane for all injections and implants. The injection site was shaved and cleaned with 70% ethanol prior to subcutaneous or intramuscular injection of a formulation. For subcutaneous (S.C.) vaccination, a 100 μl dose was delivered on a single flank of the hind limb and for intramuscular (I.M.) vaccination a 50 μl dose was delivered into the thigh muscle of the hind limb. For making R-DOTAP based vaccine formulations, R-DOTAP nanoparticles (4-6 mg/ml) in 280 mM sucrose buffer were mixed 1:1 with indicated concentrations of recombinant proteins resuspended in 280 mM sucrose buffer. For antigen-only vaccine formulations, recombinant protein was resuspended at the desired concentration in 280 mM sucrose buffer. All vaccination regimens consisted of two doses delivered at 1-4-week intervals.

### Hemagglutination Inhibition Assay:

The hemagglutination inhibition (HAI) assay was used to assess functional antibodies to the HA that are able to inhibit agglutination of guinea pig erythrocytes for H3N2 viruses, and turkey erythrocytes for H1N1 viruses. The protocols were adapted from the World Health Organization (WHO) laboratory influenza surveillance manual. Guinea pig red blood cells are frequently used to characterize contemporary A(H3N2) influenza strains that have developed a preferential binding to alpha (2,6) linked sialic acid receptors. To inactivate nonspecific inhibitors, sera samples were treated with receptor-destroying enzyme (RDE) prior to being tested. Briefly, three parts of RDE was added to one part of sera and incubated overnight at 37° C. RDE was inactivated by incubation at 56° C. for 30 min.

RDE-treated sera were diluted in a series of two-fold serial dilutions in v-bottom microtiter plates. An equal volume of each A(H3N2) virus, adjusted to approximately 8 hemagglutination units (HAU)/50 μl in the presence of 20 nM Oseltamivir carboxylate, was added to each well. The plates were covered and incubated at room temperature for 30 mins, and then 0.75% guinea pig erythrocytes in PBS were added. Prior to use, the red blood cells (RBCs) were washed twice with PBS, stored at 4° C., and used within 24 h of preparation. The plates were mixed by gentle agitation, covered, and the RBCs were allowed to settle for 1 h at room temperature. The HAI titer was determined by the reciprocal dilution of the last well that contained non-agglutinated RBCs. Positive and negative serum controls were included for each plate.

In separate assays, RDE-treated sera were diluted in a series of two-fold serial dilutions in v-bottom microtiter plates. An equal volume of each influenza virus, adjusted to approximately 8 hemagglutination units (HAU)/50 μl was added to each well. The plates were covered and incubated at room temperature for 20 mins with erythrocytes in phosphate buffered saline (PBS). Prior to use, the RBCs were washed twice with PBS, stored at 4° C., and used within 24 h of preparation. The plates were mixed by gentle agitation, covered, and the RBCs were allowed to settle for 30 mins at room temperature. The HAI titer was determined by the reciprocal dilution of the last well that contained non-agglutinated RBCs. Positive and negative serum controls were included for each plate.

All mice were negative (HAI≤1:10) for pre-existing antibodies to human influenza viruses prior to infection or vaccination, and for this study sero-protection was defined as HAI titer>1:40 and seroconversion as a 4-fold increase in titer compared to baseline, as per the WHO and European Committee for Medicinal Products to evaluate influenza vaccines. All mice were naïve and seronegative at the time of vaccination, and thus sero-conversion and sero-protection rates are interchangeable for this study.

### Mouse Challenge Experiments:

For influenza virus challenge studies, the DBA/2J mice (female, 7 to 9 weeks old) were immunized with indicated vaccine formulations intramuscularly on day 0 and day 28. On day 56, they were challenged with the H1N1 A/Brisbane/02/2018 (Bris/18) influenza virus at an 10× LD₅₀ dose of 3.6*10⁶ pfu/mouse intranasally with the volume of 50 μL. Mock vaccinated animals were inoculated intranasally with 50 uL of PBS. All animals challenged with live virus were monitored twice daily, morning and evening, for weight loss and clinical signs (labored breathing, lethargy, hunched back, ruffled fur, failure to respond to stimuli, and severe respiratory distress), for up to 14 days post infection. The body weight was tightly monitored until 14 days post-infection. Mice were humanely euthanized once they lost 20% of their original body weight or they reached clinical endpoints. Lungs in influenza challenge mice were collected from three pre-selected mice in each group on day 3 and day 6 post-infection for viral titer detection. Briefly, frozen lungs were processed and clarified supernatants containing virus were added to MDCK cells at 90% confluence and incubated for 1 hr. following this step, cells were washed and supplemented with media containing 1.6% agarose. After 72 hr incubation at 37° C., plates were processed, dried and viral plaques were enumerated plaque forming units per gram of lung tissue.

### Equipment, Software and Statistical Analysis:

Flow cytometry was performed using B.D. Symphony A3 flow cytometer equipped with BD FACSDiva™ software. All flow data was analyzed using FlowJo® version 10.0 software. Statistical analysis for all other studies were performed using GraphPad Prism 9.0 software and comparing means by a simple student's T test or by ANOVA with Tukey multiple comparison correction. Mantel-Cox test was used for survival curves.

### Example 2

Immunogenicity of Recombinant Influenza Protein Formulated with R-DOTAP Nanoparticles

The ability of R-DOTAP to enhance the immunogenicity of recombinant influenza proteins was assessed. For this, influenza nucleoprotein, or computationally optimized broadly reactive antigen (COBRA) hemagglutinins (HA) were used as vaccine antigens. In a first set of experiments, nucleoproteins were formulated with R-DOTAP nanoparticles, B6 mice were immunized with two doses of vaccine; the T cell immune response was then assessed using a validated H2-D^{b} binding CD8 T cell epitope (NP366-74: ASNENMETM, SEQ ID NO:1) and a validated I-A^{b} binding CD4 T cell epitope (NP-311-25: QVYSLIRPNENPAHK, SEQ ID NO:2).

In a second set of experiments, monovalent and bivalent vaccine (R-DOTAP-Y2NG2) containing recombinant COBRA sequences representing H1N1 hemagglutinin (Y2) and H3N2 hemagglutinin (NG2) formulated with R-DOTAP nanoparticles were prepared, BALB/cJ mice were vaccinated intramuscularly with two doses of vaccine and T cell responses were measured 7 days after a booster dose using an IFN-γ ELISpot assay.

to stimulate antigen-specific T cells in the ELISpot assay, whole COBRA proteins or overlapping peptides from A/California 07/2009 (H1N1), or A/New York/384/05(H3N2) hemagglutinins, which share consensus with COBRA sequences and are relevant to the naturally circulating influenza viruses were used.

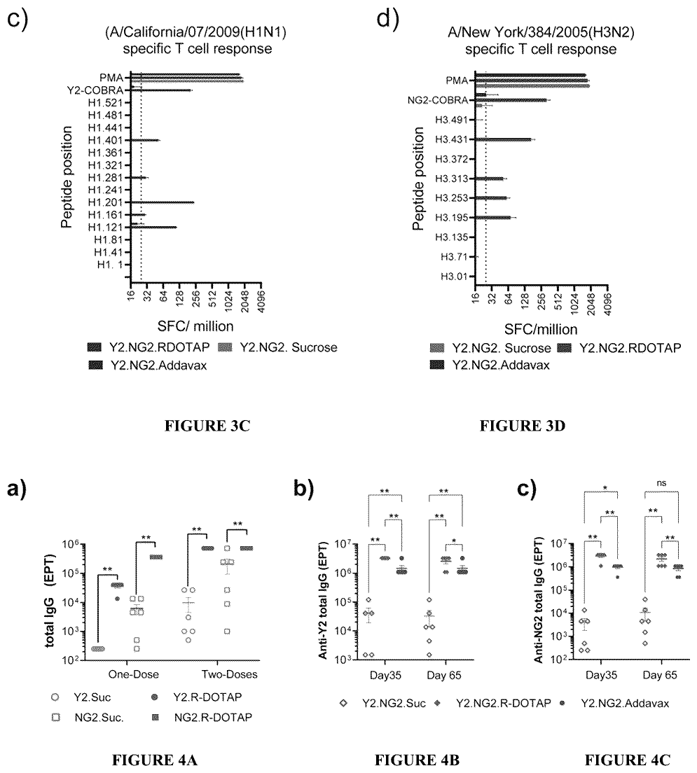

As shown in FIGS. 3A-3D, the R-DOTAP containing formulations induced strong T cell responses to both nucleoprotein and COBRA HA antigens. R-DOTAP based vaccines induced strong CD4⁺ and CD8⁺ T cell responses to nucleoprotein compared to antigen only vaccines (FIG. 3A). Similarly, mice vaccinated with R-DOTAP-Y2NG2 bivalent vaccine showed robust T cell responses to COBRA antigens (FIGS. 3B-3D). T cell immune responses induced by R-DOTAP adjuvanted formulations were significantly higher compared Addvax® adjuvanted formulations, an oil-emulsion based adjuvants system. Importantly, T cells raised against COBRA antigens recognize and respond to multiple conserved T cell epitopes presented by naturally occurring hemagglutinins from H1N1 and H3N2 strains of viruses (FIGS. 3B-3D).

### Example 3

Effect of Recombinant Influenza Protein Formulated with R-DOTAP Nanoparticles on Antigen Specific Antibody Titers

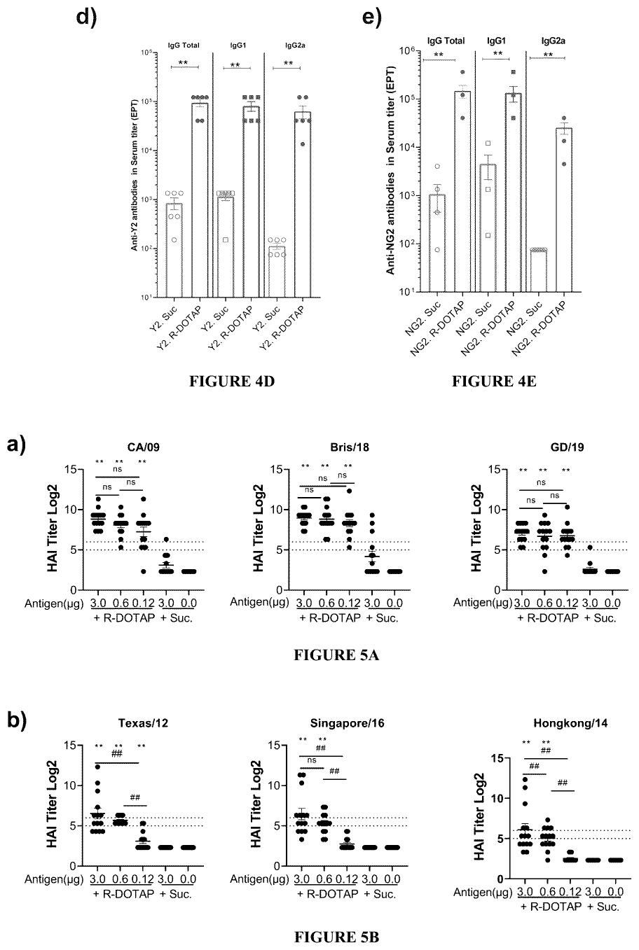

To assess antibody mediated immune responses to COBRA proteins, serum samples from vaccinated mice were analyzed for antigen specific antibody titers. A significant enhancement of antibody titers with a single injection of the R-DOTAP based formulation was observed (FIG. 4A). A second dose further increased antibody titers significantly as compared to antigen-only groups. Mixing two COBRA antigens in a single bivalent vaccine did not pose any formulation issues with R-DOTAP. The addition of NP to the bivalent vaccine was also evaluated and did not encounter any compatibility or stability limitations (data not shown).

When compared to Addavax™ adjuvanted formulation, mice vaccinated with R-DOTAP containing formulations showed higher antibody titers measured at day 35 and day 62 (FIGS. 4B and 4C), indicating that R-DOTAP induced potent antibody induction comparable to Addavax™. Dose sparing potential was next evaluated by immunizing BALB/cJ mice with varying doses of COBRA-Y2 formulated with varying doses of R-DOTAP nanoparticles and Y2-specific antibody responses were measured. Mice immunized with 0.35-3.0 μg of Y2 antigen formulated with 300 μg of R-DOTAP showed significantly increased Y2-specific total IgG titers measured 14 days after the boost vaccine (FIG. 1). Animals vaccinated with 0.35 μg or 3.0 μg showed similar IgG titers. Similarly, mice vaccinated with 50-300 μg of R-DOTAP added to the vaccine formulations induced similar elevation in total antibody titers without any significant differences between low dose and high dose of R-DOTAP (FIG. 1).

Th1 type antibody mediated immune responses play an important role in protection against viral infections. To further assess R-DOTAP induced immune response, antibody subclass titers following vaccination were measured. It was observed that R-DOTAP induced class switching, as evidenced by presence of IgG1 and IgG2a antibodies in day 35 serum samples. similar levels of IgG1 and IgG2a were observed, indicating a balanced Th1/Th2 response (FIGS. 4D and 4E).

### Example 4

Effect of Recombinant Influenza Protein Formulated with R-DOTAP Nanoparticles on the Generation of Neutralizing Antibodies

The functional ability of vaccine to elicit antibodies that block influenza virus interaction with sialic acids was next characterized. The hemagglutination inhibition (HAI) assay was used, to evaluate the generation of influenza virus neutralizing antibodies to different strains of H1N1 and H3N2 viruses.

Bivalent vaccines (R-DOTAP-Y2NG2) formulated with R-DOTAP showed significantly enhanced titers compared to antigen only vaccines (FIGS. 5A-5B) to multiple drift variants of both H1N1 and H3N2 virus strains that share consensus with the COBRA Y-2, and COBRA-NG2 respectively. Robust HAI titers above the 1:40 threshold were observed for all H1N1 viruses, even at the lowest dose (0.12 μg) of antigen tested. HAI titers against H3N2 drift variants were lower than HAI titers against H1N1 viruses, but still showed significant HAI titers above the 1:40 threshold for the 3 μg dose. As expected, very little neutralizing activity (HAI titer<1:40) of antibodies induced with antigen alone samples or from mock vaccinated mice was observed. Together these results show that vaccination with bivalent COBRA antigens formulated with R-DOTAP induced robust and balanced Th1 and Th2 antibody responses and broadly cross-reactive antibodies that can neutralize several H1N1 and H3N2 strains drift variants.

### Example 5

### R-DOTAP can Enhance Immunogenicity of Unadjuvanted Seasonal Influenza Vaccines

Whether R-DOTAP can be used to enhance immunogenicity to existing human influenza vaccines was evaluated. As a proof of concept, a trivalent inactivated influenza vaccine, Fluzone® (2011-12 formulation) consisting of a split virus hemagglutinin preparation derived from A/California/07/2009 X-179A (H1N1), A/Victoria/210/2009 X-187 (an A/Perth/16/2009-like virus) (H3N2), and B/Brisbane/60/2008 was used. Vaccine formulations were prepared by mixing 1:1 ratio of R-DOTAP nanoparticles and varying doses of Fluzone®. C57BL/6J mice were vaccinated (0.1 ml/dose) on day 0 and day 21 and blood was obtained for HAI titers on day 35.

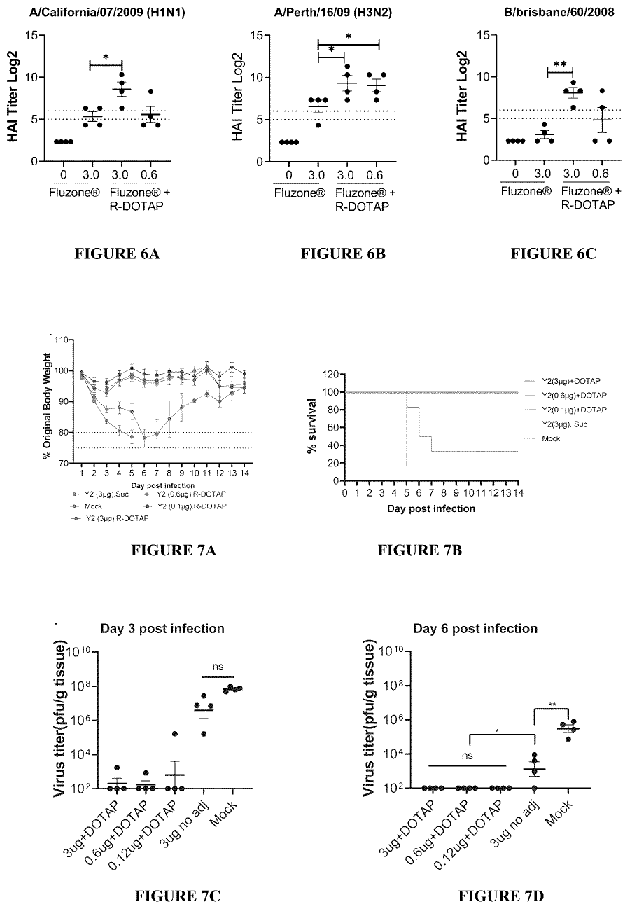

It was observed that formulating Fluzone® with R-DOTAP significantly enhanced the HAI titers to all viral strains (FIGS. 6A-6C) in vaccinated mice compared to Fluzone® only vaccinated groups. Importantly a significant dose sparing effect in R-DOTAP vaccine groups was observed.

### Example 6

Recombinant Proteins Formulated with R-DOTAP Protected Mice from Challenge Influenza Virus

To determine the protective efficacy of bivalent influenza vaccine R-DOTAP-Y2NG2 in mice, DBA/2J mice were vaccinated with formulations containing varying doses of COBRA antigens with or without R-DOTAP in a two-dose regimen followed by challenge with A/Brisbane/2/2018 (H1N1) (3.6×10⁶ pfu/dose) 28 days post vaccination. Weight loss was measured as predictor of protection from challenge.

100% of both unvaccinated mice and mice vaccinated with antigen-only (3 μg/HA) vaccine formulations rapidly lost body weight with 0 and 33% survival, respectively within 7 days (FIGS. 7A and 7B). In contrast less than 5% weight loss and 100% survival were observed in all groups vaccinated with the bivalent vaccine formulated with R-DOTAP (FIGS. 7A and 7B). Both low dose (0.12 μg/HA) and high dose (3 μg/HA) COBRA antigen formulated with R-DOTAP provided complete protection indicating a significant dose sparing effect (FIG. 7A).

The viral clearance in the lungs following challenge was also evaluated. Vaccine formulations containing R-DOTAP completely cleared influenza virus in the lungs within 3 days (FIG. 7C) and no virus was detected by day 6 (FIG. 7D). In contrast, mice receiving antigen only and unvaccinated mice showed significant viral load both at day 3 and day 6. Together, these studies demonstrated that recombinant protein vaccines containing R-DOTAP as immune modulator induced immune responses capable of protecting mice from viral infections.

### Example 7

### Discussion

Cationic lipids are excellent delivery vehicles for transporting nucleic acids and protein/peptides into cells and have been widely used in human drug delivery. However, most cationic lipids are inert and do not activate immunological signals necessary for effective immune response to vaccine antigens. Here, it was demonstrated that R-DOTAP promotes robust antibody and T cell responses to a variety of viral proteins capable of providing neutralizing activity and protection from viral challenge.

Both TEM pictures (FIGS. 2A-2C) and physical characterization data (Table 1 & FIGS. 2A-2C) showed that R-DOTAP formed uniform smooth surface spherical structures ranging around 150 nm size in sucrose buffer. Mixing antigens with the nanoparticles did not significantly alter the size and polydispersity of the formulation (table 1). Changes in the surface and zeta potential of the nanoparticles following antigen addition were observed.

TABLE 1
Particle size, polydispersity index (PDI)
and zeta potential of R-DOTAP formulations.

| | Average particle | PDI | ζ Potential (mv) |
|---|---|---|---|
| Formulation | Dia. (nm) ± Stdev | Average ± Stdev | average ± Stdev |
| R-DOTAP | 152.87 ± 0.40 | 0.10 ± 0.01 | 54.7 ± 2.10 |
| COBRA-Y2. R-DOTAP | 148.67 ± 0.70 | 0.13 ± 0.01 | 45.90 ± 1.82 |
| COBRA-NG2.R-DOTAP | 147.90 ± 1.05 | 0.11 ± 0.01 | 46.70 ± 1.31 |
| COBRA-Y2.NG2. R-DOTAP | 148.2 ± 1.13 | 0.11 ± 0.00 | 46.20 ± 0.10 |

In this study, the ability of R-DOTAP to enhance both humoral and cellular immune responses to large protein antigens using prototype vaccine formulations containing antigens derived from the respiratory virus Influenza was evaluated. By formulating R-DOTAP with the COBRA influenza antigens the potential for the R-DOTAP-COBRA vaccine to provide an effective universal flu vaccine by inducing a broadly protective immune response that may neutralize multiple strains of the flu virus was evaluated (FIGS. 5A-5B). It was demonstrated that co-formulating Influenza derived viral protein antigens with R-DOTAP significantly enhanced vaccine immunogenicity, generated robust antigen-specific cellular and antibody-mediated immune responses in mice, mediated significant antigen dose sparing, and protected vaccinated mice from influenza virus challenge.

The present results demonstrated that single component R-DOTAP nanoparticles can perform multiple tasks essential to generating broad and durable protective immune responses.

There is ample evidence that suggests CD8 T cells play a crucial role in long-term protection against highly mutating viruses such as Influenza. While most approved adjuvants for recombinant proteins effectively induce humoral and Th1 type immune responses, very few if any can induce robust and clinically effective cytotoxic CD8 T cell immune responses in humans. The current study demonstrated the ability of R-DOTAP to generate CD8 T cells to internal epitopes of large recombinant protein antigens. These T cells were polyfunctional with an effector phenotype. They were capable of producing multiple cytotoxic cytokines and persisted in vaccinated mice 28 days after a second vaccine, indicating the establishment of T cell memory responses.

It was observed that R-DOTAP containing vaccines induced both IFN-γ and IL-4 producing T cells (data not shown) and generated both IgG1 and IgG2a antibody subtypes. No strong skewing towards either the Th1 or Th2 subtype was observed. Thus, from an antibody perspective, these results indicated that R-DOTAP induced a balanced Th1/Th2 type immunity optimal for effective vaccine induced antibody responses. However, among antigen specific Th1 and CD8 T-cells, R-DOTAP promoted the development of high levels of polyfunctional cytokine secreting cells shown to be optimal in promoting viral clearance.

In summary, these studies demonstrated that protein subunit vaccines against Influenza that are based on the R-DOTAP platform induced broadly protective cellular and humoral immune responses and provide a significant dose sparing effect on the antigen. The heterogeneity of recombinant proteins capable of being formulated and administered with R-DOTAP coupled with the ability to produce multivalent vaccines suggest that R-DOTAP is an excellent candidate for use in a variety of prophylactic vaccines against infectious disease. Furthermore, it has been demonstrated that R-DOTAP is highly effective in enhancing the potency of the currently licensed seasonal influenza vaccine, further expanding the potential utility of R-DOTAP. The safety and efficacy profiles of R-DOTAP have been successfully established in human clinical trials paving the way for future trials of R-DOTAP based universal influenza vaccines.

### Example 8

### Use of a R-DOTAP Adjuvant Formulation for Bivalent Cobra H1/H3 Vaccines in a Pre-Immune Ferret Model

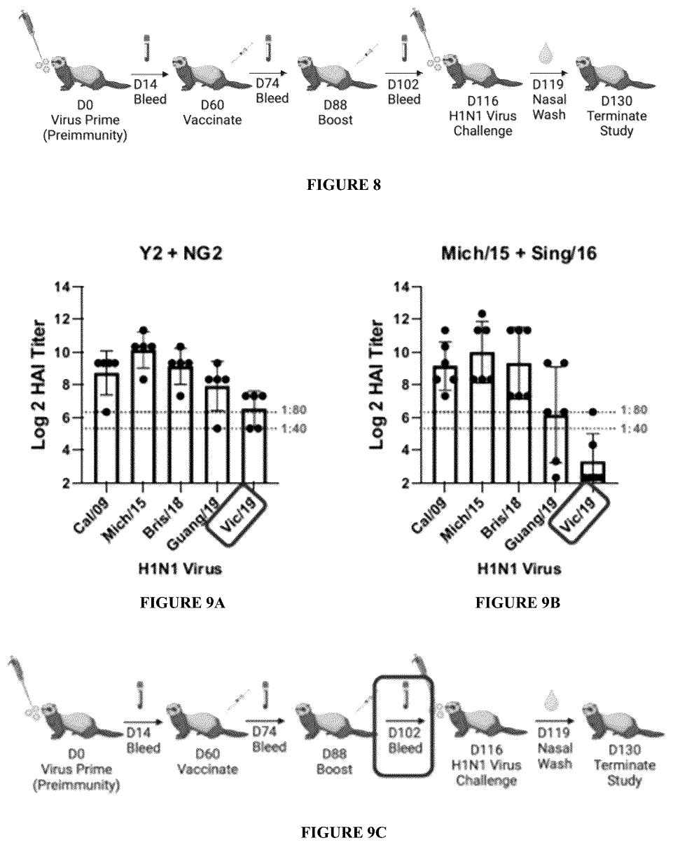

In a pre-immune ferret model, as described in FIG. 8, ferrets were primed with the virus, vaccinated, and boosted, before an influenza challenge was performed. The model mimic human response to vaccination by first infecting the ferrets with influenza viruses (H1N1—A/Singapore/6/1986—and H3N2—A/Panama/2007/1999).

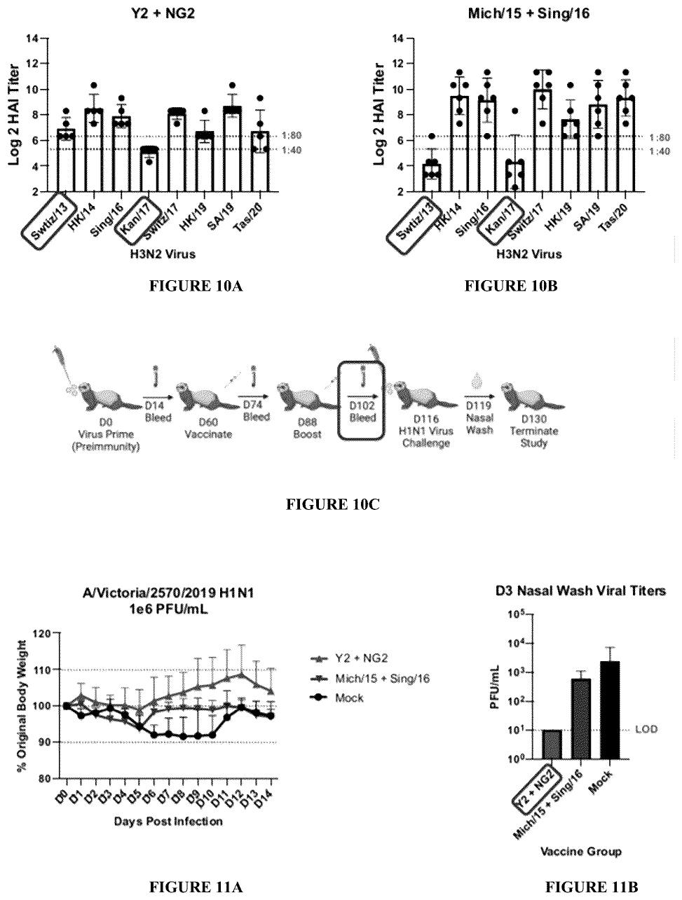

As illustrated in FIGS. 9A-9C, the H1N1 HAI response was evaluated. H1/H3 pre-immune ferrets were either vaccinated twice with Y2/NG2 rHA (15 ug), with R-DOTAP alone, or with wild type rHA and R-DOTAP, and HAI titers were measured. As illustrated in FIGS. 10A-10C, the H3N2 HAI response was also evaluated. H1/H3 pre-immune ferrets were either vaccinated twice with Y2/NG2 rHA (15 μg), with R-DOTAP alone, or with wild type rHA and R-DOTAP, and HAI titers were measured.

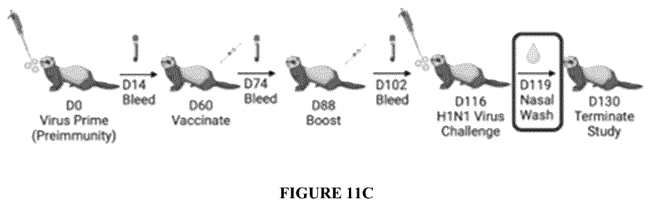

As illustrated in FIGS. 11A-11C, body weight was better maintained, and nasal wash viral titers were lower in the animals that were vaccinated with Y2/NG2 rHA.

This study demonstrated that COBRA rHA vaccines adjuvanted with Infectimune® (R-DOTAP) were capable of eliciting protective HAI antibody responses in pre-immune ferrets across panels of viruses from the last decade. It was also shown that they elicited HAI reactive antibodies against future drifted viral isolates from 2019-2020. The vaccination prevented weight loss and H1N1 viral replication in the lungs of vaccinated animals. In a population that has a more extensive pre-immune background to influenza, like humans, it is expected that these vaccines generate a more broadly reactive antibody profile due to the recall of a more diverse population of memory B cells.

Although the invention has been described with reference to the above examples, it will be understood that modifications and variations are encompassed within the spirit and scope of the invention. Accordingly, the invention is limited only by the following claims.

1. A vaccine composition comprising:
a) one or more non-naturally occurring recombinant influenza antigens; and
b) a cationic lipid.

2. The vaccine composition of claim 1, wherein the one or more non-naturally occurring recombinant influenza antigens comprise a computationally optimized broadly reactive influenza antigen (COBRA) hemagglutinins (HA).

3. The vaccine composition of claim 1, wherein the one or more non-naturally occurring recombinant influenza antigens are recombinant H1N1 and/or H3N2 hemagglutinin influenza proteins.

4. The vaccine composition of claim 1, wherein the one or more non-naturally occurring recombinant influenza antigens comprise an amino acid sequence with at least 80% sequence identity to any of SEQ ID NOs: 3-22 or combinations thereof.

5-6. (canceled)

7. The vaccine composition of claim 1, wherein the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof.

8. (canceled)

9. The vaccine composition of claim 1, wherein the one or more non-naturally occurring recombinant influenza antigens are
a) encapsulated in liposomes comprising cationic lipids,
b) mixed with preformed cationic lipid nanoparticles or
c) present as micelles separate from the cationic lipid nanoparticles.

10. (canceled)

11. The vaccine composition of claim 9, wherein the one or more non-naturally occurring recombinant influenza antigens and the preformed cationic lipid nanoparticles are mixed at a 1:1 ratio.

12. (canceled)

13. A method of inducing an immune response against an influenza virus in a subject comprising administering to the subject a vaccine composition of claim 1, thereby inducing the immune response against influenza viruses in the subject.

14-19. (canceled)

20. The method of claim 13, wherein the immune response comprises an induction of CD8⁺ effector T cells, CD4⁺ effector T cells and memory T cells.

22. The method of claim 13, wherein inducing the immune response comprises inducing the production of IgG in the subject.

23. (canceled)

24. The method of claim 13, wherein inducing the immune response comprises inducing the secretion of broadly neutralizing antibodies.

25. A method of preventing or treating an influenza infection in a subject comprising administering to the subject a vaccine composition of claim 1 thereby preventing or treating the influenza infection in the subject.

26-31. (canceled)

32. A method of enhancing immunogenicity of an influenza vaccine in a subject comprising:
a) administering an influenza vaccine to the subject; and
b) administering a cationic lipid to the subject,
thereby enhancing immunogenicity of the influenza vaccine.

33. The method of claim 32, wherein the influenza vaccine is an inactivated flu vaccine, an attenuated flu vaccine or a recombinant flu vaccine.

34. The method of claim 33, wherein the influenza vaccine is a monovalent vaccine, a bivalent vaccine, a trivalent vaccine or a quadrivalent vaccine.

35. The method of claim 32, wherein the cationic lipid is DOTAP, DDA, DOEPC, DOTMA, R-DOTAP, R-DDA, R-DOEPC, R-DOTMA, S-DOTAP, S-DDA, S-DOEPC, S-DOTMA, variations thereof or analogs thereof.

36-50. (canceled)

51. The method of claim 13, wherein the immune response comprises inducing a balanced Th1/Th2 immune response.

52-59. (canceled)

60. The method of claim 13, wherein the immune response comprises an inducing a polyfunctional CD4+/CD8+ T cell response.

61. (canceled)
